(19) United States
(12) Patent Application Publication
(10) Pub. No.: US 2004/0214804 A1
(43) Pub. Date: Oct. 28, 2004

(54) Combination of an aldosterone receptor antagonist and an anti-obesity agent

(75) Inventors: Eric Arthur Gulve, St. Louis, MO (US); Ellen Garwitz McMahon, St. Louis, MO (US)

Correspondence Address: BANNER & WITCOFF, 1001 G STREET N W, SUITE 1100, WASHINGTON, DC, 20001, US

(73) Assignee: Pharmacia Corporation, St. Louis, MO (US)

(21) Appl. No.: 10/814,870

(22) Filed: Apr. 1, 2004

Related U.S. Application Data
(60) Provisional application No. 60/465,213, filed on Apr. 25, 2003.

(51) Int. Cl. A61K 31/56

(52) U.S. Cl. 514/171

(57) ABSTRACT

A combination therapy comprising a therapeutically-effective amount of an aldosterone receptor antagonist and a therapeutically-effective amount of an anti-obesity agent is described for treatment of circulatory disorders, including cardiovascular disorders such as hypertension, congestive heart failure, cirrhosis and ascites. Preferred anti-obesity agents are those compounds having high potency and bioavailability. Preferred aldosterone receptor antagonists are 20-spiroxane steroidal compounds characterized by the presence of a 9α,11α-substituted epoxy moiety.

### CROSS-REFERENCE TO RELATED APPLICATION

This non-provisional application claims priority to provisional Application No. 60/465,213, filed Apr. 25, 2003, incorporated herein by reference in its entirety.

### FIELD OF THE INVENTION

Combinations of an aldosterone receptor antagonist and anti-obesity agents are described for use in treatment of circulatory disorders, including, but not limited to, hypertension, cardiovascular disease, renal dysfunction, cerebrovascular disease, vascular disease, retinopathy, neuropathy, hyperglycemia, hyperinsulinemia and insulin resistance, edema, endothelial dysfunction, and baroreceptor dysfunction. Of particular interest are therapies using a steroidal aldosterone receptor antagonist compound in combination with an anti-obesity agent.

### BACKGROUND OF THE INVENTION

Aldosterone

Aldosterone is the body's most potent known mineralocorticoid hormone. As connoted by the term mineralocorticoid, this steroid hormone has mineral-regulating activity. It promotes sodium (Na⁺) reabsorption not only in the kidney, but also from the lower gastrointestinal tract and salivary and sweat glands, each of which represents a classic aldosterone-responsive tissue. Aldosterone increases sodium and water reabsorption in the distal nephron and promotes potassium (K⁺) and magnesium (Mg²⁺) excretion.

Aldosterone also can produce responses in nonepithelial cells. In fact, aldosterone receptors have been recently identified in brain tissue, heart tissue and blood vessels. These aldosterone-mediated responses can have adverse consequences on the structure and function of the cardiovascular system and other tissues and organs. Hence, aldosterone can contribute to organ damage for multiple reasons.

Aldosterone Receptor Antagonists

The effect of aldosterone can be reduced through the use of an aldosterone receptor antagonist. An aldosterone receptor antagonist that is commercially available at this time is spironolactone (also known as ALDACTONE®). Spironolactone is indicated for the management of essential hypertension, primary aldosteronism, hypokalemia, and edematous conditions such as congestive heart failure, cirrhosis of the liver and nephrotic syndrome. The United States Pharmacopeia, 21^{st} Revision (16^{th} Edition), United States Pharmacopeial Convention, Inc., Rockville, Md. (1985) and each and every subsequent edition to date thereof. The administration of spironolactone to severe heart failure patients was evaluated in the Randomized Aldactone Evaluation Study (RALES). RALES was a randomized, double-blinded, placebo-controlled trial that enrolled participants who had severe heart failure and a left ventricular ejection fraction of no more than 35% and who were receiving standard therapy, including an angiotensin-converting enzyme inhibitor, a loop diuretic, and, in some cases, digoxin. The RALES subjects treated with spironolactone had a statistically significant reduction in mortality and incidence of hospitalization relative to placebo-treated subjects. New England Journal of Medicine 341, 709-717 (1999). A class of steroidal-type aldosterone receptor antagonists exemplified by epoxy-containing spirolactone derivatives is described in U.S. Pat. No. 4,559,332 issued to Grob et al. This patent describes 9α,11α-epoxy-containing spirolactone derivatives as aldosterone receptor antagonists that are useful for the treatment of hypertension, cardiac insufficiency and cirrhosis of the liver. One of the epoxy-steroidal aldosterone receptor antagonist compounds described in U.S. Pat. No. 4,559,332 is eplerenone (also known as epoxymexrenone). Eplerenone is an aldosterone receptor antagonist that has a higher specificity for the aldosterone receptor than does, for example, spironolactone.

Generic Name

The generic name of the active ingredient in INSPRA™ 25, 50 and 100 mg tablets is eplerenone. Eplerenone is also the U.S. Adopted Name (USAN) and International Nonproprietary Name (INN) for this compound. Ciba-Geigy Corporation originally referred to eplerenone by the name epoxymexrenone.

Chemical Name

The chemical name for eplerenone is pregn-4-ene-7,21-dicarboxylic acid, 9,11-epoxy-17-hydroxy-3-oxo, γ-lactone, methyl ester, (7α,11α,17α)-. This chemical name corresponds to the CAS registry name for eplerenone (the CAS registry number for eplerenone is 107724-20-9).

U.S. Pat. No. 4,559,332 identifies eplerenone by the alternative name of 9α,11α-epoxy-7α-methoxycarbonyl-20-spirox-4-ene-3,32-thione. Such “spiroxane” nomenclature is further described, for example, at column 2, line 16 through column 4, line 48 of U.S. Pat. No. 4,559,332.

WO01/95892 and WO01/95893 describe methods for the treatment of aldosterone-mediated pathogenic effects in a subject using an aldosterone receptor antagonist (including spironolactone and/or eplerenone).

WO02/09683 describes methods of using an aldosterone receptor antagonist (including eplerenone and/or spironolactone) for the treatment of inflammation in a subject.

Anti-Obesity Agents

Obesity has been linked with a multitude of health problems including, but not limited to, type 2 diabetes, hypertension, osteoarthritis, and heart disease/failure. For example, Satish Kenchaiah et al. in “Obesity and the Risk of Heart Failure” N. Engl J. Med. Vol. 347, No5 8, Aug. 1, 2002 pp 305-313, investigates a link between obesity and heart failure.

Susan Yanovski et al. in “Obesity” N. Engl J. Med. Vol. 346, No. 8, Feb. 8, 2002 pp 591-602, provide a brief history of obesity and mechanisms of action of weight loss drugs such as appetite-suppressant medications. Such medications generally include noradrenergic agents, serotonergic agents, mixed noradrenergic agents and serotonergic agents, and medications that reduce nutrient absorption. According to Brower (“Fighting fat: new drugs against obesity in the pipeline”, EMBO Reports Vol. 3, No. 7, 2002), other medications with different mechanisms of action, are in clinical trials for evaluation such as Axokine, a recombinant variant of ciliary neurotrophic factor and agonists of the 5HT2c serotonin receptor.

Combination Therapy

Therapies comprising the administration of an aldosterone receptor antagonist in combination with several other pharmacologically active compounds have been reported in the literature.

WO 96/40255, incorporated herein in its entirety, discloses a combination treatment therapy utilizing a an epoxy-steroidal aldosterone receptor antagonist and an angiotensin II antagonist for treating cardiofibrosis.

WO 96/40257, incorporated herein in its entirety, discloses a combination treatment therapy utilizing an epoxy-steroidal aldosterone receptor antagonist and an angiotensin II antagonist for treating congestive heart failure.

Perez et al., WO 00/27380, incorporated herein in its entirety, discloses a combination treatment therapy utilizing an angiotensin converting enzyme inhibitor and an aldosterone receptor antagonist for reducing morbidity and mortality resulting from cardiovascular disease.

Alexander et al., WO 00/51642, incorporated herein in its entirety, discloses a combination treatment therapy utilizing an angiotensin converting enzyme inhibitor and an epoxy-steroidal aldosterone receptor antagonist for treating cardiovascular disease.

Schuh, WO 02/09761, incorporated herein in its entirety, discloses a combination treatment therapy utilizing an epoxy-steroidal aldosterone receptor antagonist and a calcium channel blocker for treating hypertension, congestive heart failure, cirrhosis and ascites.

Rocha, WO 02/09759, incorporated herein in its entirety, discloses a combination treatment therapy utilizing an epoxy-steroidal aldosterone receptor antagonist and a cyclooxygenase-2 inhibitor for treating inflammation.

Improved drug therapies for the treatment of subjects suffering from or susceptible to a cardiovascular-related condition are highly desirable. In particular, there still is a need for drug therapies that (1) provide better control over cardiovascular-related conditions, (2) further reduce cardiovascular-related risk factors, (3) provide improved treatment and prevention of cardiovascular-related conditions, (4) are effective in a greater proportion of subjects suffering from or susceptible to a cardiovascular-related condition, particularly in those subjects who do not satisfactorily respond to conventional drug therapies, and/or (5) provide an improved side-effect profile relative to conventional drug therapies.

### BRIEF SUMMARY OF THE INVENTION

A combination therapy comprising a therapeutically-effective amount of an aldosterone receptor antagonist and a therapeutically-effective amount of an anti-obesity agent is useful to treat circulatory disorders, including cardiovascular disorders such as hypertension, cardiovascular disease, renal dysfunction, liver disease, cerebrovascular disease, vascular disease, retinopathy, neuropathy, hyperglycemia, hyperinsulinemia and insulin resistance, edema, endothelial dysfunction, and baroreceptor dysfunction.

A method for the prophylaxis or treatment of a cardiovascular-related condition, the method comprising administering to a subject susceptible to or afflicted with such condition a first amount of an aldosterone receptor antagonist and a second amount of an anti-obesity agent, wherein the first amount of the aldosterone receptor antagonist and the second amount of the anti-obesity agent together comprise a therapeutically-effective amount of the aldosterone receptor antagonist and the anti-obesity agent.

### DETAILED DESCRIPTION OF THE PREFERRED EMBODIMENTS

Unless indicated otherwise, the following definitions or terms are used throughout this specification:

The term “cardiovascular-related condition” includes, but is not limited to, circulatory disorders, cardiovascular disorders, cardiovascular diseases, coronary artery disease, hypertension, renal dysfunction, liver disease, heart failure, cerebrovascular disease, vascular disease, retinopathy, neuropathy, hyperglycemia, hyperinsulinemia, insulin resistance, edema, endothelial dysfunction, baroreceptor dysfunction, heart failure, arrhythmia, diastolic dysfunction, systolic dysfunction, ischemia, hypertrophic cardiomyopathy, sudden cardiac death, myocardial fibrosis, vascular fibrosis, impaired arterial compliance, myocardial necrotic lesions, vascular damage, myocardial infarction, left ventricular hypertrophy, decreased ejection fraction, cardiac lesions, vascular wall hypertrophy, endothelial thickening, endothelial dysfunction and fibrinoid necrosis of coronary arteries.

The terms “treat,” “treatment” or “treating” include the administration, to a person in need of or susceptible to a cardiovascular-related condition, of an amount of an aldosterone receptor antagonist and anti-obesity agent in a combination that will prevent the onset of, inhibit or reverse development of a pathological cardiovascular condition.

The terms “prevent,” “prevention” or “preventing” includes either preventing the onset of one or more clinically evident cardiovascular-related conditions altogether or preventing the onset of a preclinically evident stage of one or more cardiovascular-related conditions in individuals. This includes prophylactic treatment of those at risk of developing one or more cardiovascular-related conditions.

The phrase “therapeutically-effective” is intended to qualify the amount of the two agents given in combination which will achieve the goal of improvement in cardiovascular-related condition severity and the frequency of incidence, while avoiding adverse side effects.

The term “subject” for purposes of treatment includes any human or animal subject who is susceptible to or suffering from one or more cardiovascular-related conditions, and preferably is a human subject. The subject, for example, may be at risk due to diet, exposure to bacterial or viral infection, having common markers present, being genetically predisposed to one or more cardiovascular-related conditions, and the like.

The term “anti-obesity agent” as used herein includes, but is not limited to, any currently known weight loss agent or drug. See Remington's Pharmaceutical Sciences, 16^{th} Ed., Arthur Osol (Editor), Mack Publishing Co., Easton, Pa. (1980) and each and every subsequent edition to date thereof. See also The Merck Index, 12^{th} Edition, S. Budavari (Editor), Merck & Co., Inc., Whitehouse Station, N.J. (1996) and each and every subsequent edition to date thereof.

Aldosterone Receptor Antagonists

The term “aldosterone receptor antagonist” denotes a compound capable of binding to an aldosterone receptor, as a competitive inhibitor of the action of aldosterone itself at the receptor site, so as to modulate the receptor-mediated activity of aldosterone or other molecules that activate this receptor.

The aldosterone receptor antagonists used in the combinations and methods of the present invention generally are spirolactone-type steroidal compounds. The term “spirolactone-type” is intended to characterize a structure comprising a lactone moiety attached to a steroid nucleus, typically at the steroid “D” ring, through a spiro bond configuration. A subclass of spirolactone-type aldosterone receptor antagonist compounds consists of epoxy-steroidal aldosterone receptor antagonist compounds such as eplerenone. Another subclass of spirolactone-type antagonist compounds consists of non-epoxy-steroidal aldosterone receptor antagonist compounds such as spironolactone.

The epoxy-steroidal aldosterone receptor antagonist compounds used in the combinations and method of the present invention generally have a steroidal nucleus substituted with an epoxy-type moiety. The term “epoxy-type” moiety is intended to embrace any moiety characterized in having an oxygen atom as a bridge between two carbon atoms, examples of which include the following moieties:

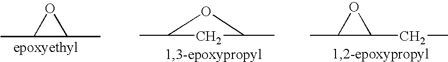

The term “steroidal”, as used in the phrase “epoxy-steroidal”, denotes a nucleus provided by a cyclopenteno-phenanthrene moiety, having the conventional “A”, “B”, “C” and “D” rings. The epoxy-type moiety may be attached to the cyclopentenophenanthrene nucleus at any attachable or substitutable positions, that is, fused to one of the rings of the steroidal nucleus or the moiety may be substituted on a ring member of the ring system. The phrase “epoxy-steroidal” is intended to embrace a steroidal nucleus having one or a plurality of epoxy-type moieties attached thereto.

Epoxy-steroidal aldosterone receptor antagonists suitable for use in the present combinations and methods include a family of compounds having an epoxy moiety fused to the “C” ring of the steroidal nucleus. Especially preferred are 20-spiroxane compounds characterized by the presence of a 9α,11α-substituted epoxy moiety. Compounds 1 through 11, below, are illustrative 9α,11α-epoxy-steroidal compounds that may be used in the present methods. A particular benefit of using epoxy-steroidal aldosterone receptor antagonists, as exemplified by eplerenone, is the high selectivity of this group of aldosterone receptor antagonists for the mineralocorticoid receptor. The superior selectivity of eplerenone results in a reduction in side effects, that can be caused by aldosterone receptor antagonists that exhibit non-selective binding to non-mineralocorticoid receptors, such as androgen or progesterone receptors.

These epoxy steroids may be prepared by procedures described in Grob et al., U.S. Pat. No. 4,559,332. Additional processes for the preparation of 9,11-epoxy steroidal compounds and their salts are disclosed in Ng et al., WO97/21720 and Ng et al., WO98/25948.

TABLE I
Aldosterone Receptor Antagonist

| Compound # | Structure | Name |
|---|---|---|
| 1 | 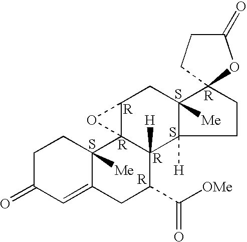 | Pregn-4-ene-7,21-dicarboxylic acid, 9,11-epoxy- 17-hydroxy-3-oxo-,γ-lactone, methyl ester, (7α,11α,17β)- |
| 2 | 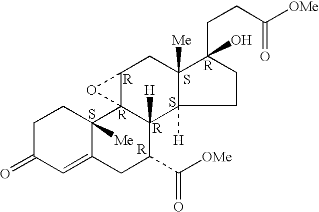 | Pregn-4-ene-7,21-dicarboxylic acid, 9,11-epoxy- 17-hydroxy-3-oxo-,dimethyl ester, (7α,11α,17β)- |
| 3 | 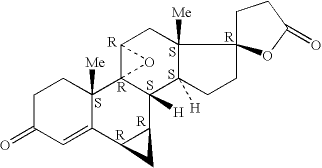 | 3′H-cyclopropa[6,7]pregna-4,6-diene-21-carboxylic acid, 9,11-epoxy-6,7-dihydro-17-hydroxy-3-oxo-, γ-lactone, (6β,7β,11α,17β)- |
| 4 | 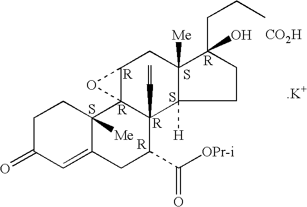 | Pregn-4-ene-7,21-dicarboxylic acid, 9,11-epoxy-17- hydroxy-3-oxo-,7-(1-methylethyl) ester, monopotassium salt, (7α,11α,17β)- |
| 5 | 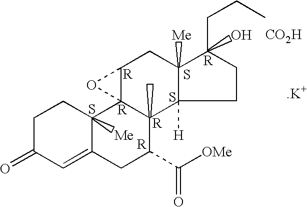 | Pregn-4-ene-7,21-dicarboxylic acid, 9,11-epoxy-17- hydroxy-3-oxo-,7-methylethyl) ester,monopotassium salt, (7α,11α,17β)- |
| 6 | 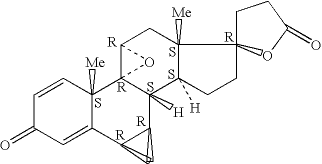 | 3′H-cyclopropa[6,7]pregna-1,4,6-triene-21-carboxylic acid, 9,11-epoxy-6,7-dihydro-17-hydroxy-3-oxo-, γ-lactone(6β,7β,11α)- |
| 7 | 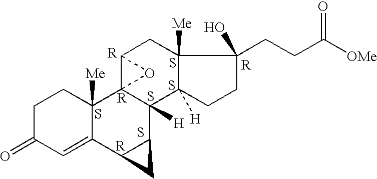 | 3′H-cyclopropa[6,7]pregna-4,6-diene-21-carboxylic acid, 9,11-epoxy-6,7-dihydro-17-hydroxy-3-oxo-, methyl ester, (6β,7β,11α,17β)- |
| 8 | 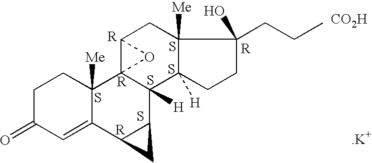 | 3′H-cyclopropa[6,7]pregna-4,6-diene-21-carboxylic acid, 9,11-epoxy-6,7-dihydro-17-hydroxy-3-oxo-, monopotassium salt, (6β,7β,11α,17β)- |
| 9 | 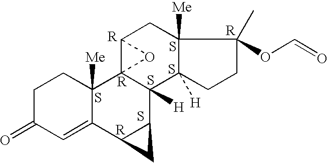 | 3′H-cyclopropa[6,7]pregna-1,4,6-triene-21-carboxylic acid, 9,11-epoxy-6,7-dihydro-17-hydroxy-3-oxo-,γ- lactone(6β,7β,11α,17β)- |
| 10 | 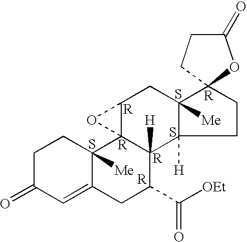 | Pregn-4-ene-7,21-dicarboxylic acid, 9,11-epoxy- 17-hydroxy-3-oxo-,γ-lactone, ethyl ester, (7α,11α,17β)- |
| 11 | 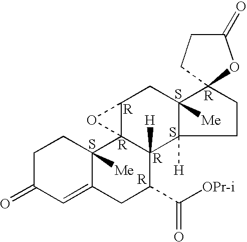 | Pregn-4-ene-7,21-dicarboxylic acid, 9,11-epoxy- 17-hydroxy-3-oxo-,γ-lactone, 1-methylethyl ester (7α,11α,17β)- |

Of particular interest is the compound eplerenone (also known as epoxymexrenone) which is compound 1 as shown above. Eplerenone is an aldosterone receptor antagonist and has a higher specificity for aldosterone receptors than does, for example, spironolactone. Selection of eplerenone as the aldosterone receptor antagonist in the present method would be beneficial to reduce certain side-effects such as gynecomastia that occur with use of aldosterone receptor antagonists having less specificity.

Non-epoxy-steroidal aldosterone receptor antagonists suitable for use in the present methods include a family of spirolactone-type compounds defined by Formula I:

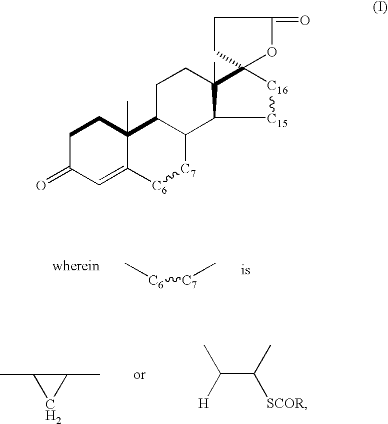

wherein R is lower alkyl of up to 5 carbon atoms, and

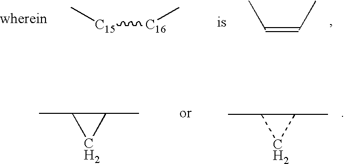

Lower alkyl residues include branched and unbranched groups, preferably methyl, ethyl and n-propyl.

Specific compounds of interest within Formula I are the following:

7α-acetylthio-3-oxo-4,15-androstadiene-[17(β-1′)-spiro-5′]perhydrofuran-2′-one;

3-oxo-7α-propionylthio-4,15-androstadiene-[17((β-1′)-spiro-5′]perhydrofuran-2′-one;

6β,7β-methylene-3-oxo4,15-androstadiene-[17((β-1′)-spiro-5′]perhydrofuran-2′-one;

15α,16α-methylene-3-oxo-4,7α-propionylthio-4-androstene[17(β-1′)-spiro-5′]perhydrofuran-2′-one;

6β,7β,15α,16α-dimethylene-3-oxo-4-androstene[17(β-1′)-spiro-5′]-perhydrofuran-2′-one;

7α-acetylthio-15β, 16β-Methylene-3-oxo-4-androstene-[17(β-1′)-spiro-5′]perhydrofuran-2′-one;

15β,16β-methylene-3-oxo-7β-propionylthio-4-androstene-[17(β-1′)-spiro-5′]perhydrofuran-2′-one; and

6 β,7β,15β,16β-dimethylene-3-oxo-4-androstene-[17(β-1′)-spiro-5′]perhydrofuran-2′-one.

Methods to make compounds of Formula I are described in U.S. Pat. No. 4,129,564 to Wiechart et al. issued on Dec. 12, 1978.

Another family of non-epoxy-steroidal compounds of interest is defined by Formula (II)

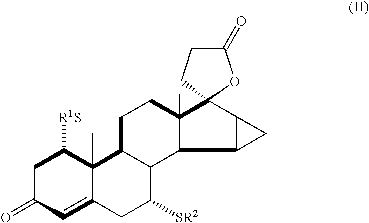

wherein R¹ is C₁₋₃-alkyl or C₁₋₃ acyl and R² is H or C₁₋₃-alkyl.

Specific compounds of interest within Formula II are the following:

1α-acetylthio-15β,16β-methylene-7α-methylthio-3-oxo-17α-pregn-4-ene-21,17-carbolactone; and

15β,16β-methylene-1α,7α-dimethylthio-3-oxo-17α-pregn-4-ene-21,17-carbolactone.

Methods to make the compounds of Formula II are described in U.S. Pat. No. 4,789,668 to Nickisch et al. which issued 6 Dec. 1988.

Yet another family of non-epoxy-steroidal compounds of interest is defined by a structure of Formula III:

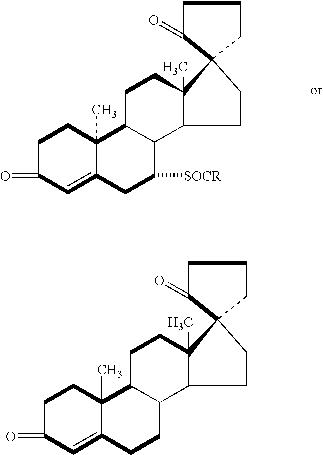

wherein R is lower alkyl, with preferred lower alkyl groups being methyl, ethyl, propyl and butyl. Specific compounds of interest include:

3β,21-dihydroxy-17α-pregna-5,15-diene-17-carboxylic acid (-lactone;

3β,21-dihydroxy-17α-pregna-5,15-diene-17-carboxylic acid (-lactone 3-acetate;

3β,21-dihydroxy-17α-pregn-5-ene-17-carboxylic acid (-lactone;

3β3,21-dihydroxy-17α-pregn-5-ene-17-carboxylic acid (-lactone 3-acetate;

21-hydroxy-3-oxo-17α-pregn-4-ene-17-carboxylic acid (-lactone;

21-hydroxy-3-oxo-17α-pregna-4,6-diene-17-carboxylic acid (-lactone;

21-hydroxy-3-oxo-17α-pregna-1,4-diene-17-carboxylic acid (-lactone;

7α-acylthio-21-hydroxy-3-oxo-17α-pregn-4-ene-17-carboxylic acid (lactone; and

7α-acetylthio-21-hydroxy-3-oxo-17α-pregn-4-ene-17-carboxylic acid (-lactone.

Methods to make the compounds of Formula III are described in U.S. Pat. No. 3,257,390 to Patchett which issued Jun. 21, 1966.

Still another family of non-epoxy-steroidal compounds of interest is represented by Formula IV:

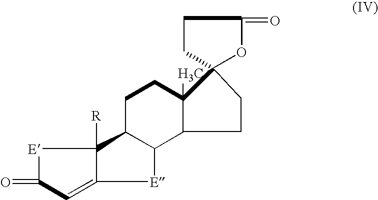

wherein E′ is selected from the group consisting of ethylene, vinylene and (lower alkanoyl)thioethylene radicals, E″ is selected from the group consisting of ethylene, vinylene, (lower alkanoyl)thioethylene and (lower alkanoyl)thiopropylene radicals; R is a methyl radical except when E′ and E″ are ethylene and (lower alkanoyl) thioethylene radicals, respectively, in which case R is selected from the group consisting of hydrogen and methyl radicals; and the selection of E′ and E″ is such that at least one (lower alkanoyl)thio radical is present.

A preferred family of non-epoxy-steroidal compounds within Formula IV is represented by Formula V:

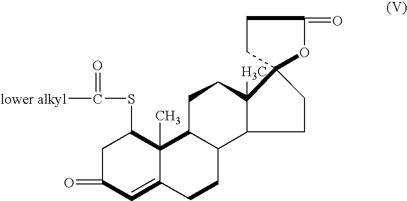

A more preferred compound of Formula V is

1-acetylthio-17α-(2-carboxyethyl)-17β-hydroxy-androst-4-en-3-one lactone.

Another preferred family of non-epoxy-steroidal compounds within Formula IV is represented by Formula VI:

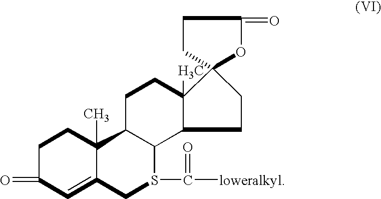

More preferred compounds within Formula VI include the following:

7α-acetylthio-17α-(2-carboxyethyl)-17β-hydroxy-androst-4-en-3-one lactone;

7β-acetylthio-17α-(2-carboxyethyl)-17β-hydroxy-androst-4-en-3-one lactone;

1α,7α-diacetylthio-17α-(2-carboxyethyl)-17β-hydroxy-androsta-4,6-dien-3-one lactone;

7α-acetylthio-17α-(2-carboxyethyl)-17β-hydroxy-androsta-1,4-dien-3-one lactone;

7α-acetylthio-17α-(2-carboxyethyl)-17β-hydroxy-19-norandrost-4-en-3-one lactone; and

7α-acetylthio-17α-(2-carboxyethyl)-17β-hydroxy-6α-methylandrost-4-en-3-one lactone;

In Formulae IV-VI, the term “alkyl” is intended to embrace linear and branched alkyl radicals containing one to about eight carbons. The term “(lower alkanoyl)thio” embraces radicals of the formula lower alkyl

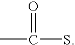

Of particular interest is the compound spironolactone having the following structure and formal name:

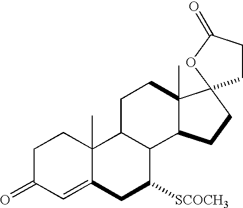

“spironolactone”: 17-hydroxy-7α-mercapto-3-oxo-17α-pregn-4-ene-21-carboxylic acid γ-lactone acetate.

Methods to make compounds of Formulae IV-VI are described in U.S. Pat. No. 3,013,012 to Cella et al. which issued Dec. 12, 1961. Spironolactone is sold by G. D. Searle & Co., Skokie, Ill., under the trademark “ALDACTONE”, in tablet dosage form at doses of 25 mg, 50 mg and 100 mg per tablet.

Another family of steroidal aldosterone receptor antagonists is exemplified by drospirenone, [6R-(6α,α,8β, 9α,10β,13β,14α,15α,16α,17β)]-1,3′,4′,6,7,8,9,10,11,12,13,14,15,16,20,21-hexadecahydro-10,13-dimethylspiro[17H-dicyclopropa[6,7:15,16]cyclopenta[a]phenanthrene-17,2′(5′H)-furan]-3,5′(2H)-dione, CAS registration number 67392-87-4. Methods to make and use drospirenone are described in patent GB 1550568 1979, priority DE 2652761 1976.

Anti-Obesity Agents

Anti-obesity agents include oral and injectable anti-obesity agents and hypoglycemia treatment agents (i.e., agents for lowering blood sugar levels, for example, anti-diabetic agents). See, for example, U.S. patent application Ser. No. 10/767,839 entitled COMBINATION OF AN ALDOSTERONE RECEPTOR ANTAGONIST AND AN ANTI-DIABETIC AGENT, filed on Jan. 30, 2004.

One embodiment includes anti-obesity agents and drugs of Table 2.

TABLE 2

| | | Reference to Source of |
|---|---|---|
| Generic Name | Mechanism of Action | Compound |
| Sibutramine | Mixed noradrenergic | Sepracor, Inc. |
| (including | and serotonergic | Tradename: Meridia |
| enantiomers) | reuptake inhibitors | (racemic mixture of sibutramine) |
| Orlistat | Gastrointestinal Lipase | Tradename: Xenical |
| | inhibitor | |

Anti-obesity agents having the following mechanisms of actions may be useful in the claimed invention: monoamine reuptake inhibitors such as sibutramine compounds; lipase inhibitors such as orlistat; CNTF variants/analogs; appetite suppressants; 5HT-2c receptor agonists; hGH fragments, e.g. AOD-9604; β₃ adrenergic receptor antagonists; Cannabinoid CB1 receptor antagonists; CCK-A receptor agonists; Neuropeptide Y (NPY-Y1 and NPY-Y5) receptor antagonists; thyroid hormone receptor beta agonists or other thyromimetic agents; glucocorticoid receptor antagonists; MCR-4 agonists; 11-beta-hydroxysteroid dehydrogenase-1 inhibitors; leptin and leptin mimetics or leptin receptor agonists; peptide YY₃-36 or analogs thereof; apolipoprotein-B/microsomal triglyceride transfer protein (apo-B/MTP) inhibitors; sympathomimetic agents; dopamine agonists such as bromocryptine; glucagon-like peptide-1 (GLP-1) analogs or GLP-1 receptor agonists; dehydroepiandrosterone (DHEA) or related analogs; melanocyte-stimulating hormone receptor analogs; melanin concentrating hormone receptor antagonists; galanin antagonists; bombesin agonists; ghrelin receptor antagonists; orexin receptor antagonists; human agouti-related proteins (AGRP) and analogs thereof; histamine-3 receptor antagonists or inverse agonists; and neuromedin U receptor agonists. See also, U.S. Non-Provisional patent application Ser. No. 10/689,381 filed on Oct. 20, 2003 and entitled “PURINE COMPOUNDS AND USES THEREOF;” U.S. Non-Provisional patent application Ser. No. 10/763,105 filed on Jan. 21, 2004 and entitled “CANNABINOID RECEPTOR LIGANDS AND USES THEREOF;” U.S. Provisional Application No. 60/518,280 filed on Nov. 7, 2003 and entitled “BICYCLIC PYRAZOLYL AND IMIDAZOLYL COMPOUNDS AND USES THEREOF;” and U.S. Non-Provisional patent application Ser. No. 10/177,858 filed on Jun. 20, 2002 and entitled “TRIAMIDE-SUBSTITUTED HETEROBICYCLIC COMPOUNDS” (same as US Publication No. 2003187053 published on Oct. 2, 2003); and U.S. Patent Application Publication No. 2004/0048912 A1 entitled “11-Beta-Hydroxysteroid Dehydrogenase 1 Inhibitors Useful for the Treatment of Diabetes, Obesity and Dyslipidemia,” filed Jun. 9, 2003 and published on Mar. 11, 2004.

One embodiment includes anti-obesity agents and drugs (in clinical development) of Table 3.

TABLE 3

| Drug Name | Company | Status | Indication | Mechanism |
|---|---|---|---|---|
| Rimonabant | Sanofi-Synthelabo | Phase III | Obesity | A selective CB1 |
| (SR- | | | | cannabinoid antagonist |
| 141716) | | | | that reduces hunger and |
| | | | | caloric intake |
| Axokine | Regeneron | Phase III | Obesity, | A modified ciliary |
| | | | obesity with | neurotrophic factor, with |
| | | | Type II | improved stability and |
| | | | diabetes | activity, that reduces |
| | | | | food intake. |
| GW-320659 | GSK | Phase II | Obesity | A dopamine/ |
| (manifaxine) | | | | norepinephrine uptake |
| | | | | inhibitor. |
| P-57 | Phytopharm | Phase II | Obesity, | Cactus-derived plant |
| (CP- | | | Type II | extract with appetite |
| 644673) | | | diabetes | suppressant activity. |
| BVT-933 | Biovitrum/GSK | Phase II | Obesity | A selective 5HT-2c |
| | | | | receptor agonist that |
| | | | | suppresses appetite. |
| HMR-1426 | Aventis | Phase II | Obesity | Peripherally acting agent |
| | | | | that delays gastric |
| | | | | emptying and increases |
| | | | | satiety, resulting in |
| | | | | reduced food intake. |
| AD 9677 | Dainippon/Takeda | Phase II | Obesity, | beta3 adrenoreceptor |
| (TAK-677) | | US | Type 2 | agonist |
| | | Phase JP | diabetes | |
| ATL-962 | Alizyme | Phase II | Obesity | A lipase inhibitor. |
| GI-181771 | GSK | Phase II | Obesity | A selective |
| | | | | cholecystokinin-A |
| | | | | receptor agonist that |
| | | | | reduces food intake |
| AOD-9604 | Metabolic | Phase II - | Obesity | A human growth |
| | | completed | | hormone fragment that |
| | | i.v.; Phase | | increases fat metabolism |
| | | II started - | | without many of |
| | | p.o. | | unwanted side effects |
| | | | | associated with hGH. |
| MLN-4760 | Millennium and | Phase I | Obesity | Selective inhibitor of a |
| | Abbott | | | carboxypeptidase. |
| L-796568 | Merck | Phase I | Obesity | beta3 adrenoreceptor |
| | | | | agonist |
| HMR-1954 | Aventis | Phase I | Obesity | 1426 back-up compound |
| SR-146131 | Sanofi-Synthelabo | Phase I | Obesity | A cholecystokinin A |
| | | | | receptor nonpeptide |
| | | | | agonist; decreases food |
| | | | | intake |
| SR-147778 | Sanofi-Synthelabo | Phase I | Obesity | Back-up to rimonabant; |
| | | | | a selective CB1 |
| | | | | cannabinoid antagonist |
| N-5984 | Nisshin Kyorin | Phase I | Obesity, | beta3 adrenoreceptor |
| | | | diabetes | agonist |
| PEG- | Regeneron | Phase I | Obesity | A longer acting form of |
| Axokine | | | | Axokine. |
| Exenatide, | Amylin | PhIII | Diabetes, | Exendin-4, An agonist |
| AC2993 | | (diabetes) | Obesity | of GLP-1 receptor |
| | Restoragen | PhIII | Diabetes, | An agonist of GLP-1 |
| | | | Obesity | receptor |
| | | | Obesity | Glucocorticoid receptor |
| | | | | antagonists |
| | Amylin | | Obesity | Peptide YY₃₋₃₆ and/or |
| | | | | analog thereof |
| | Nastech | PhI | Obesity | Peptide YY₃₋₃₆ and/or |
| | | | | analog thereof |
| | JT | Preclinical | Obesity | Microsomal triglyceride |
| | | | | transfer protein (MTP) |
| | | | | inhibitor |
| | Merck | PhII | Obesity | NPY-Y5R antagonist |
| | Shionogi | PhI | Obesity | NPY-Y5R antagonist |
| | Merck | | Obesity | MCR4-R agonist |
| | Arena | PhI | Obesity | 5HT-2c receptor agonist |
| | Merck | | Diabetes, | 11βHSD-1 inhibitor |
| | | | obesity | |
| | Biovitrum/Amgen | PhII | Diabetes, | 11βHSD-1 inhibitor |
| | | | obesity | |
| | Aventis | | Diabetes, | 5HT/NE reuptake |
| | | | obesity | inhibitor |
| | Seprecor | PhII | Obesity | 5HT/NE reuptake |
| | | | | inhibitor |
| | Tularik | Preclinical | Obesity | Leptin mimetic |
| | Aventis | | Diabetes, | 5HT/NE reuptake |
| | | | Obesity | inhibitor |

TABLE 3A
Structures

| Drug | |
|---|---|
| Name | Structure |
| Rimo- nabant (SR- 141716) | 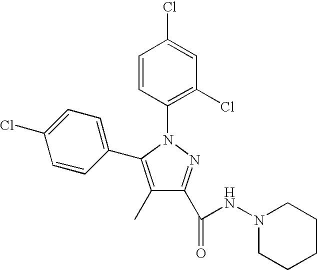 |
| P-57 (CP- 644673) | 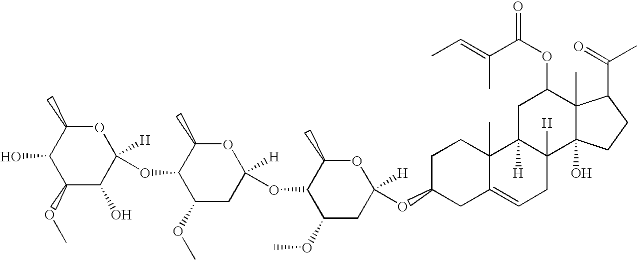 |
| Exendin- 4, AC-2993 | 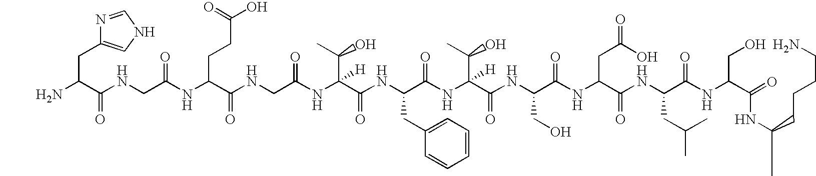 |
| | 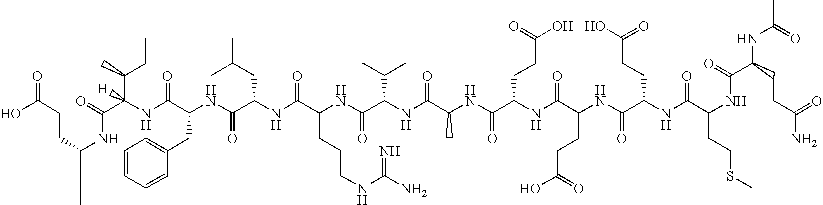 |
| | 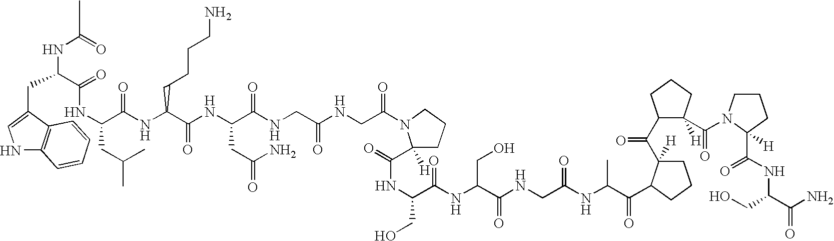 |

In one embodiment, the anti-obesity agent is selected from gastrointestinal lipase inhibitors, such as Orlistat (Xenical); and mixed norepinephrine and serotonin reuptake inhibitors, such as sibutramine (Meridia) and enantiomers thereof. Meridia is a racemic mixture of sibutramine.

In another embodiment, the aldosterone receptor antagonist is eplerenone and the anti-obesity agent is selected from Orlistat and Sibutramine.

In another embodiment, the anti-obesity agent is a ciliary neurotrophic factor or one of its related variants or analogs, such as Regeneron's Axokine; an appetite suppressant such as Pfizer/Phytopharm CP-644673/P-57; a 5-HT-2c receptor agonist, such as Biovitrum/GSK's BVT-933; a cannabinoid antagonist, such as Sanofi-Synthelabo's Rimonobant/SR-141716; a peptide or nonpeptide agonist of the cholecystokinin-A receptor such as GSK GI-181771; a peripherally acting agent that delays gastric emptying and increases satiety such as Aventis 1426; an agonist of GLP-1 receptor: GLP-1s and related analogs such as Exendin-4.

In another embodiment, the aldosterone receptor antagonist is eplerenone and the anti-obesity agent is selected from Regeneron's Axokine, Pfizer/Phytopharm CP-644673/P-57, Biovitrum/GSK's BVT-933, Sanofi-Synthelabo's Rimonobant/SR-141716, GSK GI-181771, Aventis 1426, Exendin-4.

In another embodiment, the anti-obesity agent is human growth hormone fragments such as Metabolic Pharma's AOD-9604 or any related molecule; PTP-1B inhibitors (small molecule or antisense); and DPP-IV inhibitors.

In another embodiment, the aldosterone receptor antagonist is eplerenone and the anti-obesity agent is human growth hormone fragments such as Metabolic Pharma's AOD-9604 or any related molecule; PTP-1B inhibitors (small molecule or antisense); and DPP-IV inhibitors.

In another embodiment, the anti-obesity agent is selected from neuropeptide Y1 antagonists; neuropeptide Y5 antagonists; thyroid hormone receptor beta agonists; glucocorticoid antagonists; melanocortin-4 receptor (MC-4) agonists; and adiponectin/APM1/acrp30 and related analogs.

In another embodiment, the aldosterone receptor antagonist is eplerenone and the anti-obesity agent is selected from neuropeptide Y1 antagonists; neuropeptide Y5 antagonists; thyroid hormone receptor beta agonists; glucocorticoid antagonists; melanocortin-4 receptor (MC-4) agonists; and adiponectin/APM1/acrp30 and related analogs.

In another embodiment, the anti-obesity agent is selected from selected from 11-beta-hydroxysteroid dehydrogenase-1 inhibitors; fatty acid synthase inhibitors; and acetyl CoA carboxylase inhibitors

In another embodiment, the aldosterone receptor antagonist is eplerenone and the anti-obesity agent is selected from selected from 11-beta-hydroxysteroid dehydrogenase-1 inhibitors; fatty acid synthase inhibitors; and acetyl CoA carboxylase inhibitors.

The combination therapy of the invention would be useful in treating a variety of complications of obesity states including, but not limited to, of circulatory disorders, including cardiovascular disorders, such as hypertension, congestive heart failure, myocardial fibrosis and cardiac hypertrophy. The combination therapy would also be useful with adjunctive therapies. For example, the combination therapy may be used in combination with other drugs, such as a diuretic, to aid in treatment of hypertension. The combination therapy would also be useful with adjunctive therapies comprising three or more compounds selected from one or more anti-obesity agents in combination with one or more aldosterone receptor antagonists.

In addition to the aldosterone receptor antagonist and anti-obesity agent, a third a compound may be added to the combination therapy selected from the group consisting of anti-diabetic agents, renin inhibitors, angiotensin I antagonists, angiotensin II antagonists, angiotensin converting enzyme inhibitors, alpha-adrenergic receptor blockers, beta-adrenergic receptor blockers, calcium channel blockers, endothelin receptor antagonists, endothelin converting enzymes, vasodilators, diuretics, cyclooxygenase-2 inhibitors, apical sodium bile acid transport inhibitors, cholesterol absorption inhibitors, fibrates, niacin, statins, cholesteryl ester transfer protein inhibitors, bile acid sequestrants, acyl-CoA:cholesterol acyltransferase inhibitors, anti-oxidants, vitamin E, probucol, IIb/IIIa antagonists, xemilofiban, and orbofiban.

Exemplary suitable angiotensin converting enzyme inhibitors are benazapril, captopril, cilazapril, enalapril, fosinopril, lisinopril, perindopril, quinopril, ramipril, trandolapril, and the pharmaceutically acceptable salts, esters, conjugate acids, and prodrugs thereof.

Indications

Combination therapy used to treat or prevent complications of obesity. These complications include, but are not limited to, coronary artery disease, hypertension, cardiovascular disease, renal dysfunction, cerebrovascular disease, vascular disease, hyperglycemia, hyperinsulinemia and insulin resistance, edema, endothelial dysfunction, baroreceptor dysfunction, and the like. Cardiovascular disease includes, but is not limited to, coronary artery disease, heart failure (such as congestive heart failure), arrhythmia, diastolic dysfunction (such as left ventricular diastolic dysfunction, diastolic heart failure, and impaired diastolic filling), systolic dysfunction, ischemia, sudden cardiac death, myocardial and vascular fibrosis, impaired arterial compliance, myocardial necrotic lesions, vascular damage, myocardial infarction, left ventricular hypertrophy, decreased ejection fraction, cardiac lesions, vascular wall hypertrophy, endothelial thickening, fibrinoid necrosis of coronary arteries, and the like. Renal dysfunction includes, but is not limited to, glomerulosclerosis, end-stage renal disease, diabetic nephropathy, reduced renal blood flow, increased glomerular filtration fraction, proteinuria, decreased glomerular filtration rate, decreased creatinine clearance, microalbuminuria, renal arteriopathy, ischemic lesions, thrombotic lesions, global fibrinoid necrosis, focal thrombosis of glomerular capillaries, swelling and proliferation of intracapillary (endothelial and mesangial) and/or extracapillary cells (crescents), expansion of reticulated mesangial matrix with or without significant hypercellularity, malignant nephrosclerosis (such as ischemic retraction, thrombonecrosis of capillary tufts, arteriolar fibrinoid necrosis, and thrombotic microangiopathic lesions affecting glomeruli and microvessels), and the like. Cerebrovascular disease includes, but is not limited to stroke. Vascular disease includes, but is not limited to, thrombotic vascular disease (such as mural fibrinoid necrosis, extravasation and fragmentation of red blood cells, and luminal and/or mural thrombosis), proliferative arteriopathy (such as swollen myointimal cells surrounded by mucinous extracellular matrix and nodular thickening), atherosclerosis, decreased vascular compliance (such as stiffness, reduced ventricular compliance and reduced vascular compliance), endothelial dysfunction, and the like. Edema includes, but is not limited to, peripheral tissue edema, hepatic congestion, splenic congestion, liver ascites, respiratory or lung congestion, and the like. Hyperglycemia, hyperinsulinemia and insulin resistance include, but are not limited to, insulin resistance, Type I diabetes mellitus, Type II diabetes mellitus, glucose intolerance, pre-diabetic state, metabolic syndrome, and the like.

The combination therapy is particularly useful for complications selected from the group consisting of coronary artery disease, hypertension, cardiovascular disease, renal dysfunction, edema, cerebrovascular disease, and hyperglycemia, hyperinsulinemia and insulin resistance; more preferably, the pathogenic effects are selected from the group consisting of coronary artery disease, hypertension, cardiovascular disease, stroke, and Type II diabetes mellitus; and still more preferably, the pathogenic effects are selected from the group consisting of coronary artery disease, hypertension, heart failure (particularly heart failure post myocardial infarction), left ventricular hypertrophy, and stroke.

In one embodiment of the present invention, therefore, the method comprises administering a therapeutically-effective amount of one or more epoxy-steroidal compounds that are aldosterone receptor antagonists to treat or prevent one or more aldosterone receptor-mediated pathogenic effects in a human subject suffering from or susceptible to the pathogenic effect or effects, wherein the subject has an elevated, normal, or sub-normal endogenous aldosterone level and/or an enhanced response or sensitivity to aldosterone. The pathogenic effect or effects preferably are selected from the group consisting of hypertension, cardiovascular disease, cerebrovascular disease, and Type II diabetes mellitus; and more preferably, the pathogenic effects are selected from the group consisting of hypertension, heart failure (particularly heart failure post myocardial infarction), left ventricular hypertrophy, and stroke. The epoxy-steroidal compound preferably is eplerenone. The subject of the treatment or prophylaxis preferably is an individual having salt sensitivity and/or an elevated dietary sodium intake.

Patients or Subjects of Treatment

The patients or subjects of the treatment or prophylaxis of the invention include subjects susceptible to obesity, clinically diagnosed with obesity, or subjects with subjects with metabolic syndrome, including, but not limited to Syndrome X. Body mass index (BMI) equals a person's weight in kilograms divided by the square of height in meters (BMI=kg/m²). Subjects having a BMI of 25 and up are generally considered to be overweight. Subjects having a BMI of over 30 are generally considered to be obese, with degrees of obesity increasing with BMI of over 35, and over 40.

Metabolic syndrome symptoms can include obesity/abdominal obesity, frank diabetes, hypertension, dyslipidemia (hypertriglyceridemia, low HDL-cholesterol, and/or smaller and more atherogenic forms of LDL-cholesterol, etc.), insulin resistance, microalbuminuria, and a hypercoagulable state. The patients or subjects may also include those having salt sensitivity and/or an elevated dietary sodium intake. See for example, Earl S. Ford, et al., JAMA, Jan. 16, 2002, Vol. 287, No. 3, pp 356-359.

Definitions

1881 The term “hydrido” denotes a single hydrogen atom (H). This hydrido group may be attached, for example, to an oxygen atom to form a hydroxyl group; or, as another example, one hydrido group may be attached to a carbon atom to form a

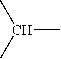

group; or, as another example, two hydrido atoms may be attached to a carbon atom to form a —CH₂— group. Where the term “alkyl” is used, either alone or within other terms such as “haloalkyl” and “hydroxyalkyl”, the term “alkyl” embraces linear or branched radicals having one to about twenty carbon atoms or, preferably, one to about twelve carbon atoms. More preferred alkyl radicals are “lower alkyl” radicals having one to about ten carbon atoms. Most preferred are lower alkyl radicals having one to about five carbon atoms. The term “cycloalkyl” embraces cyclic radicals having three to about ten ring carbon atoms, preferably three to about six carbon atoms, such as cyclopropyl, cyclobutyl, cyclopentyl and cyclohexyl. The term “haloalkyl” embraces radicals wherein any one or more of the alkyl carbon atoms is substituted with one or more halo groups, preferably selected from bromo, chloro and fluoro. Specifically embraced by the term “haloalkyl” are monohaloalkyl, dihaloalkyl and polyhaloalkyl groups. A monohaloalkyl group, for example, may have either a bromo, a chloro, or a fluoro atom within the group. Dihaloalkyl and polyhaloalkyl groups may be substituted with two or more of the same halo groups, or may have a combination of different halo groups. A dihaloalkyl group, for example, may have two fluoro atoms, such as difluoromethyl and difluorobutyl groups, or two chloro atoms, such as a dichloromethyl group, or one fluoro atom and one chloro atom, such as a fluoro-chloromethyl group. Examples of a polyhaloalkyl are trifluoromethyl, 1,1-difluoroethyl, 2,2,2-trifluoroethyl, perfluoroethyl and 2,2,3,3-tetrafluoropropyl groups. The term “difluoroalkyl” embraces alkyl groups having two fluoro atoms substituted on any one or two of the alkyl group carbon atoms. The terms “alkylol” and “hydroxyalkyl” embrace linear or branched alkyl groups having one to about ten carbon atoms any one of which may be substituted with one or more hydroxyl groups. The term “alkenyl” embraces linear or branched radicals having two to about twenty carbon atoms, preferably three to about ten carbon atoms, and containing at least one carbon-carbon double bond, which carbon-carbon double bond may have either cis or trans geometry within the alkenyl moiety. The term “alkynyl” embraces linear or branched radicals having two to about twenty carbon atoms, preferably two to about ten carbon atoms, and containing at least one carbon-carbon triple bond. The term “cycloalkenyl” embraces cyclic radicals having three to about ten ring carbon atoms including one or more double bonds involving adjacent ring carbons. The terms “alkoxy” and “alkoxyalkyl” embrace linear or branched oxy-containing radicals each having alkyl portions of one to about ten carbon atoms, such as methoxy group. The term “alkoxyalkyl” also embraces alkyl radicals having two or more alkoxy groups attached to the alkyl radical, that is, to form monoalkoxyalkyl and dialkoxyalkyl groups. The “alkoxy” or “alkoxyalkyl” radicals may be further substituted with one or more halo atoms, such as fluoro, chloro or bromo, to provide haloalkoxy or haloalkoxyalkyl groups. The term “alkylthio” embraces radicals containing a linear or branched alkyl group, of one to about ten carbon atoms attached to a divalent sulfur atom, such as a methylthio group. Preferred aryl groups are those consisting of one, two, or three benzene rings. The term “aryl” embraces aromatic radicals such as phenyl, naphthyl and biphenyl. The term “aralkyl” embraces aryl-substituted alkyl radicals such as benzyl, diphenylmethyl, triphenylmethyl, phenyl-ethyl, phenylbutyl and diphenylethyl. The terms “benzyl” and “phenylmethyl” are interchangeable. The terms “phenalkyl” and “phenylalkyl” are interchangeable. An example of “phenalkyl” is “phenethyl” which is interchangeable with “phenylethyl”. The terms “alkylaryl”, “alkoxyaryl” and “haloaryl” denote, respectively, the substitution of one or more “alkyl”, “alkoxy” and “halo” groups, respectively, substituted on an “aryl” nucleus, such as a phenyl moiety. The terms “aryloxy” and “arylthio” denote radicals respectively, provided by aryl groups having an oxygen or sulfur atom through which the radical is attached to a nucleus, examples of which are phenoxy and phenylthio. The terms “sulfinyl” and “sulfonyl”, whether used alone or linked to other terms, denotes, respectively, divalent radicals SO and SO₂. The term “aralkoxy”, alone or within another term, embraces an aryl group attached to an alkoxy group to form, for example, benzyloxy. The term “acyl” whether used alone, or within a term such as acyloxy, denotes a radical provided by the residue after removal of hydroxyl from an organic acid, examples of such radical being acetyl and benzoyl. “Lower alkanoyl” is an example of a more preferred sub-class of acyl. The term “amido” denotes a radical consisting of nitrogen atom attached to a carbonyl group, which radical may be further substituted in the manner described herein. The term “monoalkylaminocarbonyl” is interchangeable with “N-alkylamido”. The term “dialkylaminocarbonyl” is interchangeable with “N,N-dialkylamido”. The term “alkenylalkyl” denotes a radical having a double-bond unsaturation site between two carbons, and which radical may consist of only two carbons or may be further substituted with alkyl groups which may optionally contain additional double-bond unsaturation. The term “heteroaryl”, where not otherwise defined before, embraces aromatic ring systems containing one or two heteroatoms selected from oxygen, nitrogen and sulfur in a ring system having five or six ring members, examples of which are thienyl, furanyl, pyridinyl, thiazolyl, pyrimidyl and isoxazolyl. Such heteroaryl may be attached as a substituent through a carbon atom of the heteroaryl ring system, or may be attached through a carbon atom of a moiety substituted on a heteroaryl ring-member carbon atom, for example, through the methylene substituent of imidazolemethyl moiety. Also, such heteroaryl may be attached through a ring nitrogen atom as long as aromaticity of the heteroaryl moiety is preserved after attachment. For any of the foregoing defined radicals, preferred radicals are those containing from one to about ten carbon atoms.

Specific examples of alkyl groups are methyl, ethyl, n-propyl, isopropyl, n-butyl, sec-butyl, isobutyl, tert-butyl, n-pentyl, isopentyl, methylbutyl, dimethylbutyl and neopentyl. Typical alkenyl and alkynyl groups may have one unsaturated bond, such as an allyl group, or may have a plurality of unsaturated bonds, with such plurality of bonds either adjacent, such as allene-type structures, or in conjugation, or separated by several saturated carbons.

Racemates. Stereoisomers, and Salts thereof.

As noted above, the aldosterone receptor antagonists and anti-obesity agents useful in the present combination therapy also may include the racemates and stereoisomers, such as diastereomers and enantiomers, of such agents. Such stereoisomers can be prepared and separated using conventional techniques, either by reacting enantiomeric starting materials, or by separating isomers of compounds of the present invention. Isomers may include geometric isomers, for example cis isomers or trans isomers across a double bond. All such isomers are contemplated among the compounds of the present invention. Such isomers may be used in either pure form or in admixture with those agents described above. Such stereoisomers can be prepared using conventional techniques, either by reacting enantiomeric starting materials, or by separating isomers of compounds of the present invention.

Isomers may include geometric isomers, for example cis-isomers or trans-isomers across a double bond. All such isomers are contemplated among the compounds useful in the present invention.

The compounds useful in the present invention as discussed below include their salts, solvates and prodrugs. The compounds useful in the present invention also include tautomers. The term “pharmaceutically-acceptable salts” embraces salts commonly used to form alkali metal salts and to form addition salts of free acids or free bases. The nature of the salt is not critical, provided that it is pharmaceutically-acceptable. Suitable pharmaceutically-acceptable acid addition salts may be prepared from an inorganic acid or from an organic acid. Examples of such inorganic acids are hydrochloric, hydrobromic, hydroiodic, nitric, carbonic, sulfuric and phosphoric acid. Appropriate organic acids may be selected from aliphatic, cycloaliphatic, aromatic, araliphatic, heterocyclic, carboxylic and sulfonic classes of organic acids, examples of which are formic, acetic, propionic, succinic, glycolic, gluconic, lactic, malic, tartaric, citric, ascorbic, glucuronic, maleic, fumaric, pyruvic, aspartic, glutamic, benzoic, anthranilic, p-hydroxybenzoic, salicyclic, phenylacetic, mandelic, embonic (pamoic), methansulfonic, ethanesulfonic, 2-hydroxyethanesulfonic, pantothenic, benzenesulfonic, toluenesulfonic, sulfanilic, mesylic, cyclohexylaminosulfonic, stearic, algenic, beta-hydroxybutyric, malonic, galactaric and galacturonic acid. Suitable pharmaceutically-acceptable base addition salts include metallic salts made from aluminium, calcium, lithium, magnesium, potassium, sodium and zinc or organic salts made from N,N′-dibenzylethylenediamine, chloroprocaine, choline, diethanolamine, ethylenediamine, meglumine (N-methylglucamine) and procaine. All of these salts may be prepared by conventional means from the corresponding compound by reacting, for example, the appropriate acid or base with such compound.

Mechanism of Action

Increased body mass index (BMI, in kg/m², an index of obesity) is associated with increased risk of coronary heart disease (CHD). Compared to lean men, men with a BMI of 25-29 kg/m² have a 70% greater risk of CHD and men with a BMI of 29-33 kg/m² have almost a three-fold greater risk of CHD (Rimm E B, Stampfer M J, Giovannucci E, Ascherio A, Spiegelman D, Colditz G A and W C Willet. Body size and fat distribution as predictors of coronary heart disease among middle-aged and older US men. Am J Epidemiol 141(2):1117-1127, 1995). It is believed that the association between BMI and CHD is due to the hyperinsulinemia associated with obesity and the elevated blood pressure which is more prevalent in overweight compared to lean individuals. Multiple large epidemiological studies have suggested that insulin resistance, even in the absence of frank diabetes, is a predictor of coronary artery disease (J E Reusch, Am. J. Cardiol. 90(suppl): 19G-26G, 2002).

In general these studies have shown a relationship between plasma insulin levels (a surrogate marker of insulin resistance) and cardiovascular disease. For example, the Helsinki Policemen Study demonstrated that the incidence of cardiovascular mortality, nonfatal MI, and other cardiovascular events was associated with increasing plasma insulin levels. Blood pressure levels are also positively and continuously related to the risks of CHD and stroke (1999 World Health Organization —International Society of Hypertension Guidelines for the Management of Hypertension. J Hypertension 17:151-183, 1999). For stroke and CHD, there is no lower level of blood pressure identified below which the risks of stroke and CHD do not continue to decline.

Hypertension and obesity are two important components of the cluster of risk factors that is currently known as The Metabolic Syndrome. Hyperinsulinemia, dyslipidemia (hypertriglyceridemia, reduced HDL-cholesterol, and/or smaller and more atherogenic forms of LDL-cholesterol), microalbuminuria, and a hypercoagulable state are additional components. The presence of the Metabolic Syndrome increases the risk for developing cardiovascular disease and cardiovascular mortality (Isomaa B, Almgren P, Tuomi T, Forsen B, Lahti K, Nissen M et al. Cardiovascular morbidity and mortality associated with Metabolic Syndrome. Diabetes Care 24: 683-689, 2001). The prevalence of CHD, MI, and stroke are all substantially elevated in individuals displaying the Metabolic Syndrome, compared to those without the syndrome. Insulin resistance, hypertension, and microalbuminuria are amongst the important predictors of cardiovascular morbidity and mortality in this syndrome.

Given the independent effects of obesity (and the associated hyperinsulinemia and insulin resistance) and those of hypertension to accelerate the development of cardiovascular disease, it is anticipated that combining the effects of aldosterone receptor blockade with anti-obesity therapy should ameliorate the progression of cardiovascular disease in obese hypertensives compared to the effects of either treatment alone. Combination therapy with an aldosterone blocker and an anti-obesity agent, having beneficial effects on the macrovasculature, as well as the microvasculature, should be clinically important in obese individuals.

The progression of atherosclerotic disease is believed to be due in part to a proinflammatory state (Ridker P, Nader R, Rose L, Buring J E, Cook N R. Comparison of C-reactive protein and low-density lipoprotein cholesterol levels in the prediction of first cardiovascular events. N Engl J Med 347(20): 1557-1565). It is now also recognized that states of obesity and insulin resistance are characterized by increased oxidative stress and inflammation. The proinflammatory state in obesity may contribute to the enhanced rate of atherosclerosis since a significant relationship between plasma C-reactive protein (CRP) levels and measures of central adiposity is found in men with atherogenic dyslipidemia (Lemieux I, Pascot A, Prud'homme D, Almeras N, Bogaty P, Nadeau A et al. Elevated C-reactive protein. Another component of the atherothromboic profile of abdominal obesity. Arterioscler Thromb Vasc Biol 21:961-967, 2001). In addition, weight loss reduces CRP levels (Tchernof A, Nolan A, Sites C K, Ades P A and Poehlman E T. Weight loss reduces C-reactive protein levels in obese postmenopausal women. Circulation 105:564-569, 2002), possibly through a reduction in adipose tissue secretion of IL-6, the primary regulator of CRP synthesis by the liver (Heinrich P C, Castell J V and T Andus. Interleukin-6 and the acute phase response. Biochem J 265:621-636, 1990). Some of the beneficial cardiovascular effects of the lipid-lowering statin class of drugs (inhibitors of HMG-CoA reductase) and the antidiabetic PPARgamma agonists have been ascribed to their additional anti-inflammatory actions (P Dandona and A Aljada, Am. J. Cardiol. 90(suppl): 27G-33G, 2002). Therefore, applicants would expect weight loss with an anti-obesity agent to reduce markers of inflammation in obese patients.

Aldosterone antagonism has been shown to have pronounced anti-inflammatory effects in the heart and kidney. In the aldosterone salt model of aldosterone-mediated cardiovascular disease, aldosterone antagonism reduces cytokines (COX-2, MCP-1, IL-6, IL-1b and osteopontin) in various tissues (Rocha R, Rudolph A E, Frierdich G E, Nachowiak N, Kekec B K, Blomme E A G, et al. Aldosterone induces a vascular inflammatory phenotype in the rat heart. Amer. J. Physiol. 283:H1802-H1810, 2002). Therefore the combination of weight loss resulting from treatment with an anti-obesity agent and an aldosterone receptor antagonist may have beneficial effects to reduce inflammation and significantly attenuate the development of atherosclerosis in obese individuals.

In recent years it has become evident that adipose tissue synthesizes and secretes a number of proteins that have actions in the vasculature, such as plasminogen activator inhibitor-1 (Sobel B E. Effects of glycemic control and other determinants on vascular disease in Type 2 diabetes. Am. J. Med 113(6A):12S-22S, 2002), angiotensinogen (Engeli S, Negrel R, Sharma A M. Physiology and pathophysiology of the adipose tissue renin-angiotensin system. Hypertension 35:1270-1277, 2000), and adiponectin (Yamauchi T, Kamon J, Waki H, Imai Y, Shimozawa N, Hioki K et al. Globular adiponectin protected ob/ob mice from diabetes and apoE-deficient mice from atherosclerosis. J. Biol. Chem 278(4):2461-2468, 2003). Adipose tissue expression of these proteins is dysregulated in obesity. Furthermore, adipose tissue appears to express the key components of the renin-angiotensin system. It has been hypothesized that adipose tissue production of angiotensin may contribute to hypertension often seen in obesity and Type II diabetes (Gorzelniak K, Engeli S, Janke J, Luft F C and A M Sharma. Hormonal regulation of the human adipose-tissue renin-angiotensin system: relationship to obesity and hypertension. J Hypertens. 20:965-973, 2002).

Moreover, plasma aldosterone is correlated positively with measures of visceral obesity and insulin resistance (Goodfriend T L, Egan B M and D E Kelley. Plasma aldosterone, plasma lipoproteins, obesity and insulin resistance in humans. Prostaglandins, Leukot Essent Fatty Acids 60(5&6):401-406, 1999), suggesting that visceral fat stimulates adrenal steroidogenesis. This hypothesis is supported by the observation that weight reduction of as little as 5 kg reduces blood pressure in a large proportion of obese hypertensive individuals and also has a beneficial effect on associated risk factors such as insulin resistance, diabetes, dyslipidemia and left ventricular hypertrophy (LVH) (Reid C M, Dart A M, Dwar E M and G L Jennings. Interactions between the effects of exercise and weight loss on risk factors, cardiovascular haemodynamics and left ventricular structure in overweight subjects. J Hypertens 12:291-301, 1991). Weight loss has been shown to increase plasma levels of adiponectin (Hotta K, Funahashi T, Arita Y, Takahashi M, Matsuda M et al. Plasma concentrations of a novel, adipose-specific protein, adiponectin, in Type 2 diabetic patients. Arterioscler. Thromb. Vasc. Biol. 20:1595-1599, 2000; Yang W-S, Lee W-J, Funahashi T, Tanaka S, Matsuzawa Y, Chao C-L, Chen C-L, Tai T-Y, and L-M Chuang. Weight reduction increases plasma levels of an adipose-derived anti-inflammatory protein, adiponectin. J. Clin. Endocrinol. Metabiii. 86:3815-3819, 2001), which in turn has been shown to have beneficial effects on the vasculature (Matsuda M, Shimomura I, Sata M, Arita Y, Nishida M, et al. Role of adiponectin in preventing vascular stenosis. The missing link of adipo-vascular axis. J. Biol. Chem. 277:37487-37491, 2002; Okamoto Y, Kihara S, Ouchi N, Nishida M, Arita Y, et al. Adiponectin reduces atherosclerosis in apolipoprotein E-deficient mice. Circulation 106:2767-2770, 2002) that may act in additive or synergistic fashion to the vascular effects of aldosterone receptor antagonists. Aldosterone receptor antagonists also reduce LVH and improve microalbuminuria in hypertensive patients, suggesting that the combination of weight loss with an anti-obesity agent and an aldosterone receptor antagonist could provide significant benefit to reduce cardiovascular disease in obese hypertensives (Pitt B. The 4E Study: Eplerenone, enalapril and eplerenone/enalapril combination in patients with LVH. J Am Coll Card 40:14, 2002; Epstein M, Buckalew V, Martinez F, Altamirano J, Roniker B, Kleiman J, Krause S. Antiproteinuric efficacy of eplerenone, enalapril, and eplerenone/enalapril combination therapy in diabetic hypertensives with microalbuminuria. Amer J Hypertens 15:24A, 2002).

Left ventricular mass is known to be a powerful and independent predictor of cardiovascular morbidity and mortality (for example, see D. Levy et al., Prognostic implications of echocardiographically determined left ventricular mass in the Framingham heart study, New Engl. J. Med., Vol. 322, pp. 1561-1566 (1990); F. Gueyffier et al., Identification of risk factors in hypertensive patients: contribution of randomized controlled trials through an individual patient database, Ann. Int. Med., Vol. 100, pp. e88-e94 (1999)). Body mass index (BMI) and systolic blood pressure in turn are important and independent predictors of left ventricular mass (for example, see I. W. Hammond et al., Relation of blood pressure and body build to left ventricular mass in normotensive and hypertensive employed adults, J. Am. Coll. Cardiol., Vol. 12, pp. 996-1004 (1988)). The effect of systolic blood pressure to increase left ventricular mass is amplified by obesity, such that the slope of this graphical relationship is increased in obese subjects with elevated body mass index (see J. S. Gottdiener et al., Importance of obesity, race, and age to the cardiac structural and functional effects of hypertension, J. Am. Coll. Cardiol., Vol. 24, pp. 1492-1498 (1994)). Cardiac function is further compromised in obesity by the fact that obese patients suffer from a reduction in left ventricular peak filling rate, even when compared with lean patients characterized by a similar degree of hypertension (for example, see E. Grossman et al., Left ventricular filling in the systemic hypertension of obesity, Am. J. Cardiol., Vol. 68, pp. 57-60 (1991)). Given these independent effects of obesity and hypertension on left ventricular size and function, we propose that addition of an anti-obesity agent to an aldosterone receptor antagonist should have important benefits in the obese hypertensive population.

In the population of patients who undergo unilateral nephrectomy, a subset of these individuals develops proteinuria and progressive renal failure. Patients who had previously undergone unilateral nephrectomy for a wide variety of medical reasons were evaluated in an attempt to identify characteristics of the subpopulations that did subsequently develop renal dysfunction. In this study of obese uninephrectomized patients, obesity was identified as the major risk factor characterizing the subgroup of patients that developed proteinuria and/or progressive renal failure (these types of patients with a single kidney can ill-afford to have the remaining kidney malfunction); Kaplan-Meier estimated probability curves for proteinuria and renal function predicted a dramatic worsening of outcomes over 10-20 years in patients with elevated BMI (see M. Praga et al., Influence of obesity on the appearance of proteinuria and renal insufficiency after unilateral nephrectomy, Kidney International, vol. 58, pp. 2111-2118 (2000)). Based on these findings, the combination of an anti-obesity agent with an aldosterone receptor antagonist would have clinically important benefits in the prevention of progressive deterioration of renal function in patients who have undergone unilateral nephrectomy.

Advantages of Combination Therapy

The selected aldosterone receptor antagonists and anti-obesity agent of the present invention act in combination to provide more than an additive benefit. For example, administration of an aldosterone receptor antagonist and an anti-obesity agent combination can result in the near-simultaneous reduction in pathogenic effects of multiple risk factors for coronary heart disease and atherosclerosis. For example, drug combinations may reduce or counteract several risk factors for atherosclerosis, such as high aldosterone levels, high blood pressure, endothelial dysfunction, insulin resistance, elevated plasma triglycerides, vascular inflammation, a prothrombotic state, etc. Both weight loss and aldosterone antagonism reduce vascular inflammation and it is now believed that vascular inflammation may play a significant role in the development of atherosclerosis, especially plaque rupture (Atherosclerosis—an inflammatory disease. N Eng J Med 340(2):115-126, 1999). The blood pressure lowering effects of weight reduction may be enhanced by reduction of sodium intake in older hypertensive subjects (Whelton P K, Appel L J, Espeland M A, Applegate W B, Ettinger W H, Kostis J B et al., for TONE Collaborative Research Group. Sodium reduction and weight loss in the treatment of hypertension in older persons: a randomized controlled trial of nonpharmacologic interventions in the Elderly (TONE). JAMA 279:839-846, 1998). This effect would likely be reproduced with administration of an aldosterone receptor antagonist (which decreases aldosterone-mediated sodium retention) and an anti-obesity agent to cause the weight loss.

The methods of this invention also provide for the effective prophylaxis and/or treatment of pathological conditions with reduced side effects compared to conventional methods known in the art. For example, administration of anti-obesity agents can result in side effects such as, but not limited to, increases in blood pressure, nausea, and gastrointestinal distress. Reduction of the anti-obesity agent doses in the present combination therapy below conventional monotherapeutic doses will minimize, or even eliminate, the side-effect profile associated with the present combination therapy relative to the side-effect profiles associated with, for example, monotherapeutic administration of anti-obesity agents. The side effects associated with anti-obesity agents typically are dose-dependent and, thus, their incidence increases at higher doses. Accordingly, lower effective doses of anti-obesity agents will result in fewer side effects than seen with higher doses of anti-obesity agents in monotherapy or decrease the severity of such side effects.

Other benefits of the present combination therapy include, but are not limited to, the use of a selected group of aldosterone receptor antagonists that provide a relatively quick onset of therapeutic effect and a relatively long duration of action. For example, a single dose of one of the selected aldosterone receptor antagonists may stay associated with the aldosterone receptor in a manner that can provide a sustained blockade of mineralocorticoid receptor activation. Because complications of obesity result from chronic exposure to risk factors such as hypertension and hyperinsulinemia, more sustained reduction in risk factor profiles is expected to enhance the treatment effect. Another benefit of the present combination therapy includes, but is not limited to, the use of a selected group of aldosterone receptor antagonists, such as the epoxy-steroidal aldosterone receptor antagonists exemplified by eplerenone, which act as highly selective aldosterone receptor antagonists, with reduced side effects that can be caused by aldosterone receptor antagonists that exhibit non-selective binding to non-mineralocorticoid receptors, such as androgen or progesterone receptors. The use of selective aldosterone blockers is expected to reduce the incidence of side effects such as impotence, gynecomastia, breast pain and menstrual irregularities.

Further benefits of the present combination therapy include, but are not limited to, the use of the methods of this invention to treat individuals who belong to one or more specific racial or ethnic groups that are particularly responsive to the disclosed therapeutic regimens. Thus, for example, individuals of African, native American, or Hispanic ancestry may particularly benefit from the combination therapy of an aldosterone receptor antagonist and an anti-obesity agent to treat or prevent cardiovascular disease. The incidence and prevalence of obesity (Flegal K M, Carroll M D, Kuczmarski R J, Johnson C L. Overweight and obesity in the United States: prevalence and trends, 1960-1994. Int. J. Obes. 22:39-47, 1998) and stroke and hypertensive renal disease is higher in African Americans, Hispanic Americans, and Native Americans compared to non-Hispanic whites (Lee E J, Cowan L D, Welty T K, Sievers M, Howard W J, Oopik A., et al. All-cause mortality and cardiovascular disease mortality in three American Indian populations, aged 45-74 years, 1984-1988: The Strong Heart Study. Am. J. Epidemiol. 147:995-1008, 1998; Broderick J, Brott T, Kothari R, Miller R, Khoury J, Pancioli A, et al. The First Greater Cincinnati/Northern Kentucky Stroke Study: preliminary first-ever and total incidence rates of stroke among blacks. Stroke 29:415-421, 1998; Klag M J, Whelton P K, Randall B L, Neaton J D, Brancati FL, Stamler J. End-stage renal disease in African-American and white men. JAMA 277:1293-1298). Because aldosterone receptor blockade is more efficacious in controlling hypertension in some of these same racial/ethnic groups, e.g. in African Americans (Pratt J H, Flack J M, Wright J T, Patron A, Roniker B, Kuse-Hamilton J, Krause S. Efficacy and tolerability of eplerenone and losartan in hypertensive black patients and white patients. Am J Hypertens 15(4):213A) it is reasonable to expect that combination therapy will be more efficacious in controlling cardiovascular disease progression and the associated morbidity and mortality in the same groups.

Kits

The present invention further comprises kits that are suitable for use in performing the methods of treatment and/or prevention described above. In one embodiment, the kit contains a first dosage form comprising one or more of the aldosterone receptor antagonists identified in Table 1 and a second dosage form comprising one or more of the anti-obesity agents and agents used in treating the symptoms and conditions associated with obesity identified above in quantities sufficient to carry out the methods of the present invention. Preferably, the first dosage form and the second dosage form together comprise a therapeutically effective amount of the above-identified receptor antagonists and agents for the treatment or prevention of cardiovascular disease(s) and/or disorder(s).

In another embodiment, the kit contains a first dosage form comprising the aldosterone receptor antagonist spironolactone and a second dosage form comprising an anti-obesity agent and agents used in treating the symptoms and conditions associated with obesity identified above in quantities sufficient to carry out the methods of the present invention.

In another embodiment, the kit contains a first dosage form comprising the aldosterone receptor antagonist eplerenone and a second dosage form comprising an anti-obesity agent and agents used in treating the symptoms and conditions associated with obesity identified above in quantities sufficient to carry out the methods of the present invention.

Biological Evaluation

In order to determine the probable effectiveness of a combination therapy for obesity and related conditions and symptoms, it is important to determine the potency of components in several assays. Accordingly, in Assay “A”, the activity of an anti-obesity agent can be determined. In Assay “B”, a method is described for evaluating a combination therapy of the invention, namely, anti-obesity agent and an epoxy-steroidal aldosterone receptor antagonist. The efficacy of the individual drugs, eplerenone, and anti-obesity agent, and efficacy of these drugs given together at various doses, are evaluated in rodent models of hypertension and diabetes and related conditions and symptoms.

Therapy Protocols

Preclinical and clinical evaluation of a combination of eplerenone and an anti-obesity agent include, for example, blood pressure measurements, renal function measurements, and glycemic control measurements (plasma glucose, HbA1C, and insulin). Clinical trials for testing drug therapies for subjects with obesity and obesity related symptoms are well known. Clinical trials that can be modified to test the efficacy of the instant combination in treating obesity are described in, for example, Kelley, David E. MD, et al. “Clinical Efficacy of Orlistat Therapy in Overweight and Obese Patients With Insulin-Treated Type 2 Diabetes: A 1-year randomized controlled trial,” Diabetes Care, Volume 25(6), June 2002, pp 1033-1041; Gadde, Kishmore M. MD, “Zonisamide for Weight Loss in Obese Adults: A Randomized Controlled Trial,” JAMA, Vol 289, No. 14, Apr. 9, 2003, pp 1820-1825; and Gokcel A. et al., “Evaluation of the Safety and Efficacy of Sibutramine, Orlistat and Metformin in the Treatment of Obesity,” Diabetes. Obesity and Metabolism, vol. 4, 2002, pp 49-55.

Preclinical Trials

Animal Models: A number of different animal models of obesity, insulin resistance and diabetes are known that also display features of diabetic complications. For example, db/db mice (e.g. M. P. Cohen et al., Exp. Nephrol. 4: 166-171, 1996) and KKAy mice (K Ina et al., Diabetes Research and Clinical Practice 44: 1-8, 1999) are spontaneously obese and diabetic, and develop hypertriglyceridemia, hypercholesterolemia and renal complications reminiscent of diabetic nephropathy such as albuminuria. Fatty Zucker (fa/fa) rats are obese, insulin resistant and hypertensive, and hypertension can be exacerbated by placing animals on a high salt diet (S H Carlson et al., Hypertension 35 (1, Part 2) (Supplement):403, 2000). The Spontaneous Hypertension Heart Failure (SHHF) rat is obese, insulin-resistant, hyperlipidemic, and develops hypertension and heart failure (S. A. McCune et al., Renal and heart function in the SHHF/Mcc-cp rat. In: E Shafrir (editor): Frontiers in diabetes research. Lessons from animal diabetes III. Smith Gordon, London, 1990, pp. 397-401). Rogerio B. de Paula et al., “Aldosterone Antagonism Attenuates Obesity-Induced Hypertension and Glomerular Hyperfiltration,” Hypertension, January 2004, pp. 41-47., incorporated by reference in its entirety, provides protocols and data obtained from studying aldosterone receptor antagonist, in particular eplerenone, in mediating changes in renal function and mean arterial pressure during the development of dietary-induced obesity in dogs. Eplerenone markedly attenuated the glomerular hyperfiltration, sodium retention, and hypertension associated with chronic dietary-induced obesity, indicating that aldosterone plays an important role in the pathogenesis of obesity hypertension.

Clinical Trials

In addition, clinical trials can be used to evaluate aldosterone receptor antagonist therapy in humans. Numerous examples of such therapeutic tests have been published, including those of the RALES 003 study described in American Journal of Cardiology 78, 902-907 (1996) and the RALES 004 study described in New England Journal of Medicine 341, 709-717 (1999).

Clinical trials used to evaluate anti-obesity agents in humans have also been published. See, for example, M. Krempf et al., “Weight reduction and long-term maintenance after 18 months treatment with orlistat for obesity,” International Journal of Obesity (2003) 27, 591-597; A. Halpern et al., “Evaluation of Efficacy, Reliability, and Tolerability of Sibutramine in Obese Patients, with an Echocardiographic Study,” Rev. Hosp. Clin. Fac. Med. S. Paulo, 57(3), 89-102 (2002); FDA Label for Orlistat (Xenical) NDA 20-766/X-018; and FDA Label for Sibutramine (Meridia).

After a baseline anti-obesity therapy, patients would be treated with or without eplerenone. The results would be evaluated to determine whether eplerenone addition to anti-obesity therapy reduced complications more than anti-obesity therapy alone. Measures of efficacy would include proteinuria (urinary albumin-to-creatinine ratio), blood pressure, plasma glucose and insulin, and HbA1c.

Administration

Administration of the anti-obesity agent and the aldosterone receptor antagonist may take place sequentially in separate formulations, or may be accomplished by simultaneous administration in a single formulation or separate formulations. Administration may be accomplished by oral route, or by intravenous, intramuscular or subcutaneous injections. The formulation may be in the form of a bolus, or in the form of aqueous or non-aqueous isotonic sterile injection solutions or suspensions. These solutions and suspensions may be prepared from sterile powders or granules having one or more pharmaceutically-acceptable carriers or diluents, or a binder such as gelatin or hydroxypropyl-methyl cellulose, together with one or more of a lubricant, preservative, surface-active or dispersing agent.

Typically, the aldosterone receptor antagonist is administered in a daily dose ranging from about 0.1 to about 2000 mg, and the anti-obesity agent is administered in a daily dose ranging from about 0.1 to about 1000 mg. If included, the angiotensin converting enzyme inhibitor is administered in a daily dose ranging from about 0.1 to about 1000 mg.

For oral administration, the pharmaceutical composition may be in the form of, for example, a tablet, capsule, suspension or liquid. The pharmaceutical composition is preferably made in the form of a dosage unit containing a particular amount of the active ingredient. Examples of such dosage units are tablets or capsules. These may with advantage contain an amount of each active ingredient from about 1 to about 250 mg, preferably from about 25 to about 150 mg. A suitable daily dose for a mammal may vary widely depending on the condition of the patient and other factors. However, a dose of from about 0.01 to about 30 mg/kg body weight, particularly from about 1 to about 15 mg/kg body weight, may be appropriate.

The active ingredients may also be administered by injection as a composition wherein, for example, saline, dextrose or water may be used as a suitable carrier. A suitable daily dose of each active component is from about 0.01 to about 15 mg/kg body weight injected per day in multiple doses depending on the disease being treated. A preferred daily dose would be from about 1 to about 10 mg/kg body weight. Compounds indicated for prophylactic therapy will preferably be administered in a daily dose generally in a range from about 0.1 mg to about 15 mg per kilogram of body weight per day. A more preferred dosage will be a range from about 1 mg to about 15 mg per kilogram of body weight. Most preferred is a dosage in a range from about 1 to about 10 mg per kilogram of body weight per day. A suitable dose can be administered, in multiple sub-doses per day. These sub-doses may be administered in unit dosage forms. Typically, a dose or sub-dose may contain from about 1 mg to about 100 mg of active compound per unit dosage form. A more preferred dosage will contain from about 2 mg to about 50 mg of active compound per unit dosage form. Most preferred is a dosage form containing from about 3 mg to about 25 mg of active compound per unit dose.

In combination therapy, the anti-obesity agent may be present in a range of doses, depending on the particular agent used, inherent potency, bioavailabilty and metabolic lability of the composition and whether it has been formulated for immediate release or extended release. Non-limiting examples of dosage ranges for specific anti-obesity agents are listed below:

| COMPOUND | DOSAGE FORM | STRENGTH RANGE |
|---|---|---|
| Sibutramine | | 5-15 mg/day |
| Orlistat | | 120 mg 3 times/day |

In combination therapy, the aldosterone receptor antagonist may be present in an amount in a range from about 5 mg to about 400 mg, and the anti-obesity agent may be present in an amount in a range from about 1 mg to about 200 mg, which represents aldosterone receptor antagonist-to-anti-obesity agent ratios ranging from about 400:1 to about 1:40.

In a preferred combination therapy, the aldosterone receptor antagonist may be present in an amount in a range from about 10 mg to about 200 mg, and the anti-obesity agent may be present in an amount in a range from about 5 mg to about 100 mg, which represents aldosterone receptor antagonist-to-anti-obesity agent ratios ranging from about 40:1 to about 1:10.

In a more preferred combination therapy, the aldosterone receptor antagonist may be present in an amount in a range from about 20 mg to about 100 mg, and the anti-obesity agent may be present in an amount in a range from about 10 mg to about 80 mg, which represents aldosterone receptor antagonist-to-anti-obesity agent ratios ranging from about 10:1 to about 1:4.

The dosage regimen for treating a disease condition with the combination therapy of this invention is selected in accordance with a variety of factors, including the type, age, weight, sex and medical condition of the patient, the severity of the disease, the route of administration, and the particular compound employed, and thus may vary widely.

For therapeutic purposes, the active components of this combination therapy invention are ordinarily combined with one or more adjuvants appropriate to the indicated route of administration. If administered per os (i.e., by mouth), the components may be admixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol, and then tableted or encapsulated for convenient administration. Such capsules or tablets may contain a controlled-release formulation as may be provided in a dispersion of active compound in hydroxypropylmethyl cellulose. Formulations for parenteral administration may be in the form of aqueous or non-aqueous isotonic sterile injection solutions or suspensions. These solutions and suspensions may be prepared from sterile powders or granules having one or more of the carriers or diluents mentioned for use in the formulations for oral administration. The components may be dissolved in water, polyethylene glycol, propylene glycol, ethanol, corn oil, cottonseed oil, peanut oil, sesame oil, benzyl alcohol, sodium chloride, and/or various buffers. Other adjuvants and modes of administration are well and widely known in the pharmaceutical art. See Remington's Pharmaceutical Sciences, 16^{th} Ed., Arthur Osol (Editor), Mack Publishing Co., Easton, Pa. (1980) and each and every subsequent edition to date thereof. See also The Merck Index, 12^{th} Edition, S. Budavari (Editor), Merck & Co., Inc., Whitehouse Station, N.J. (1996) and each and every subsequent edition to date thereof.

The present invention further comprises kits that are suitable for use in performing the methods of treatment and/or prophylaxis described above. In one embodiment, the kit contains a first dosage form comprising one or more of the epoxy-steroidal aldosterone receptor antagonists previously identified and a second dosage form comprising one or more of the anti-obesity agents and agents used in treating the symptoms and conditions associated with obesity identified in Tables 2-3 in quantities sufficient to carry out the methods of the present invention. Preferably, the first dosage form and the second dosage form together comprise a therapeutically effective amount of the inhibitors.

Crystalline Forms of Active Compounds

Crystalline forms that are easily handled, reproducible in form, easily prepared, stable, and which are non-hygroscopic have been identified for the aldosterone antagonist eplerenone. These include Form H (Form I), Form L (Form II), various crystalline solvates and amorphous eplerenone. These forms, methods to make these forms, and use of these forms in preparing compositions and medicaments, are disclosed in Barton et al., WO 01/41535 and Barton et al., WO 01/42272;

incorporated herein in their entirety.

In one embodiment of the present invention, the aldosterone receptor antagonist employed comprises Form L eplerenone.

In another embodiment of the present invention, the aldosterone receptor antagonist employed comprises Form H eplerenone.

While the invention has been described with respect to specific examples including presently preferred modes of carrying out the invention, those skilled in the art will appreciate that there are numerous variations and permutations of the above described systems and techniques that fall within the spirit and scope of the invention.

Additional Embodiments

1. A method for the prophylaxis or treatment of a cardiovascular-related condition, the method comprising administering to a subject susceptible to or afflicted with such condition a first amount of an aldosterone receptor antagonist and a second amount of an anti-obesity agent,

wherein the first amount of the aldosterone receptor antagonist and the second amount of the anti-obesity agent together comprise a therapeutically-effective amount of the aldosterone receptor antagonist and anti-obesity agent.

2. The method of Embodiment 1 wherein the cardiovascular-related condition is selected from the group consisting of coronary artery disease, hypertension, cardiovascular disease, renal dysfunction, liver disease, cerebrovascular disease, vascular disease, retinopathy, neuropathy, hyperglycemia, hyperinsulinemia, insulin resistance, edema, endothelial dysfunction, and baroreceptor dysfunction.

3. The method of Embodiment 1 wherein the cardiovascular-related condition is hypertension.

4. The method of Embodiment 1 wherein the cardiovascular-related condition is cardiovascular disease.

5. The method of Embodiment 4 wherein the cardiovascular disease is selected from the group consisting of heart failure, arrhythmia, diastolic dysfunction, systolic dysfunction, ischemia, hypertrophic cardiomyopathy, sudden cardiac death, myocardial fibrosis, vascular fibrosis, impaired arterial compliance, myocardial necrotic lesions, vascular damage, myocardial infarction, left ventricular hypertrophy, decreased ejection fraction, cardiac lesions, vascular wall hypertrophy, endothelial thickening, and fibrinoid necrosis of coronary arteries.

6. The method of Embodiment 4 wherein the cardiovascular disease is heart failure.

7. The method of Embodiment 1 wherein the cardiovascular-related condition is renal dysfunction.

8. The method of Embodiment 7 wherein the renal dysfunction is selected from the group consisting of glomerulosclerosis, end-stage renal disease, diabetic nephropathy, reduced renal blood flow, increased glomerular filtration fraction, proteinuria, decreased glomerular filtration rate, decreased creatinine clearance, microalbuminuria, renal arteriopathy, ischemic lesions, thrombotic lesions, global fibrinoid necrosis, focal thrombosis of glomerular capillaries, swelling and proliferation of intracapillary cells, swelling and proliferation of extracapillary cells, expansion of reticulated mesangial matrix with or without significant hypercellularity, and malignant nephrosclerosis.

9. The method of Embodiment 1 wherein the cardiovascular-related condition is cerebrovascular disease.

10. The method of Embodiment 9 wherein the cerebrovascular disease is stroke.

11. The method of Embodiment 1 wherein the cardiovascular-related condition is vascular disease.

12. The method of Embodiment 11 wherein the vascular disease is selected from the group consisting of thrombotic vascular disease, proliferative arteriopathy, atherosclerosis, decreased vascular compliance, and endothelial dysfunction.

13. The method of Embodiment 1 wherein the cardiovascular-related condition is edema.

14. The method of Embodiment 13 wherein the edema is selected from the group consisting of peripheral tissue edema, hepatic congestion, splenic congestion, liver ascites, respiratory congestion, and lung congestion.

15. The method of Embodiment 1 wherein the cardiovascular-related condition is hyperglycemia, hyperinsulinemia, or insulin resistance.

16. The method of Embodiment 15 wherein the hyperglycemia, hyperinsulinemia or insulin resistance is selected from the group consisting of Type I diabetes mellitus, Type II diabetes mellitus, glucose resistance, pre-diabetic state, and metabolic syndrome.

17. The method of Embodiment 1 wherein the cardiovascular-related condition is selected from the group consisting of coronary heart disease, hypertension, cardiovascular disease, stroke, and Type II diabetes mellitus.

18. The method of Embodiment 17 wherein the cardiovascular-related condition is selected from the group consisting of coronary heart disease, hypertension, heart failure, left ventricular hypertrophy and stroke.

19. The method of Embodiment 1 wherein the aldosterone receptor antagonist is an epoxy-steroidal-type compound characterized in having a 9α,11α-substituted epoxy moiety.

20. The method of Embodiment 1 wherein the aldosterone receptor antagonist is eplerenone.

21. The method of Embodiment 1 wherein the aldosterone receptor antagonist is a spirolactone-type compound.

22. The method of Embodiment 1 wherein the aldosterone receptor antagonist is spironolactone.

23. The method of Embodiment 1 wherein the aldosterone receptor antagonist is selected from the group consisting of:

pregn-4-ene-7,21-dicarboxylic acid, 9,11-epoxy-17-hydroxy-3-oxo,γ-lactone, methyl ester, (7α,11α,17α)-;

pregn-4-ene-7,21-dicarboxylic acid, 9,11-epoxy-17-hydroxy-3-oxo-dimethyl ester,(7α,11α, 17α)-;

3′H-cyclopropa(6,7) pregna-4,6-diene-21-carboxylic acid, 9,11-epoxy-6,7-dihydro-17-hydroxy-3-oxo-γ-lactone, (6β,7β,11β,17β)-;

pregn-4-ene-7,21-dicarboxylic acid, 9,11-epoxy-17-hydroxy-3-oxo-,7-(1-methylethyl) ester, monopotassium salt,(7α,11α,17α)-;

pregn-4-ene-7,21-dicarboxylic acid, 9,11,-epoxy-17-hydroxy-3-oxo-,7-methyl ester, monopotassium salt, (7α,11α,17α)-;

3′H-cyclopropa(6,7)pregna-1,4,6-triene-21-carboxylic acid, 9,11-epoxy-6,7-dihydro-17-hydroxy-3-oxo-,-γ-actone(6α,7α,11α)-;

3′H-cyclopropa(6,7)pregna-4,6-diene-21-carboxylic acid, 9,11-epoxy-6,7-dihydro-17-hydroxy-3-oxo-, methyl ester, (6α,7α,11α,17α)-;

3′H-cyclopropa(6,7)pregna-4,6-diene-21-carboxylic acid, 9,11-epoxy-6,7-dihydro-17-hydroxy-3-oxo-, monopotassium salt, (6α,7α,11α,17α)-;

3′H-cyclopropa(6,7)pregna-4,6-diene-21-carboxylic acid, 9,11-epoxy-6,7-dihydro-17-hydroxy-3-oxo-, γ-lactone, (6α,7α,11α,17α)-;

pregn-4-ene-7,21-dicarboxylic acid, 9,11-epoxy-17-hydroxy-3-oxo-,γ-lactone, ethyl ester, (7α,11α,17α)-; and

pregn-4-ene-7,21-dicarboxylic acid, 9,11-epoxy-17-hydroxy-3-oxo-,γ-lactone, 1-methylethyl ester, (7α,11α,17α)-.

24. The method of Embodiment 1 wherein the anti-obesity agent is selected from the group consisting of gastrointestinal lipase inhibitors, mixed norepinephrine and serotonin reuptake inhibitors, and the pharmaceutically acceptable salts, esters, conjugate acids, and prodrugs thereof.

25. The method of Embodiment 24 wherein the aldosterone receptor antagonist is eplerenone.

26. The method of Embodiment 1 wherein the anti-obesity agent is selected from the group consisting of orlistat and sibutramine and the pharmaceutically acceptable salts, esters, conjugate acids, and prodrugs thereof.

27. The method of Embodiment 26 wherein the aldosterone receptor antagonist is eplerenone.

28. The method of Embodiment 1 wherein the anti-obesity agent is selected from the group consisting of a ciliary neurotrophic factor or one of its related variants or analogs; an appetite suppressant; a 5-HT-2c receptor agonist; a cannabinoid antagonist; a peptide or nonpeptide agonist of the cholecystokinin-A receptor; a peripherally acting agent that delays gastric emptying and increases satiety; an agonist of GLP-1 receptor: GLP-1s and related analogs, and the pharmaceutically acceptable salts, esters, conjugate acids, and prodrugs thereof.

29. The method of Embodiment 28 wherein the aldosterone receptor antagonist is eplerenone.

30. The method of Embodiment 1 wherein the anti-obesity agent is selected from the group consisting of Regeneron's Axokine, Pfizer/Phytopharm CP-644673/P-57, Biovitrum/GSK's BVT-933, Sanofi-Synthelabo's Rimonabant/SR-141716, GSK GI-181771, Aventis 1426, Exendin-4.

31. The method of Embodiment 30 wherein the aldosterone receptor antagonist is eplerenone.

32. The method of Embodiment 1 wherein the anti-obesity agent is selected from the group consisting of human growth hormone fragments; PTP-1B inhibitors; DPP-IV inhibitors, and the pharmaceutically acceptable salts, esters, conjugate acids, and prodrugs thereof.

33. The method of Embodiment 33 wherein the aldosterone receptor antagonist is eplerenone.

34. The method of Embodiment 1 wherein the anti-obesity agent is selected from the group consisting of neuropeptide Y1 antagonists; neuropeptide Y5 antagonists; thyroid hormone receptor beta agonists; glucocorticoid antagonists; melanocortin-4 receptor (MC-4) agonists; adiponectin/APM1/acrp30 and related analogs, and the pharmaceutically acceptable salts, esters, conjugate acids, and prodrugs thereof.

35. The method of Embodiment 34 wherein the aldosterone receptor antagonist is eplerenone.

36. The method of Embodiment 1 the anti-obesity agent is selected from the group consisting of 11-beta-hydroxysteroid dehydrogenase-1 inhibitors; fatty acid synthase inhibitors; acetyl CoA carboxylase inhibitors, and the pharmaceutically acceptable salts, esters, conjugate acids, and prodrugs thereof.

37. The method of Embodiment 36 wherein the aldosterone receptor antagonist is eplerenone.

38. The method of Embodiment 1 wherein the aldosterone receptor antagonist and the anti-obesity agent are administered in a sequential manner.

39. The method of Embodiment 1 wherein the aldosterone receptor antagonist and the neutral endopeptidase inhibitor are administered in a substantially simultaneous manner.

40. The method of Embodiment 1 wherein the aldosterone receptor antagonist is administered in a daily dose ranging from about 0.1 to about 2000 mg, and the anti-obesity agent is administered in a daily dose ranging from about 0.1 to about 1000 mg.

41. The method of Embodiment 1 wherein the first amount of the aldosterone receptor antagonist produces no substantial diuretic or anti-hypertensive effect in a subject.

42. The method of Embodiment 1 further comprising administering a third amount of a compound selected from the group consisting of renin inhibitors, angiotensin I antagonists, angiotensin II antagonists, angiotensin converting enzyme inhibitors, alpha-adrenergic receptor blockers, beta-adrenergic receptor blockers, calcium channel blockers, endothelin receptor antagonists, endothelin converting enzymes, vasodilators, diuretics, cyclooxygenase-2 inhibitors, apical sodium bile acid transport inhibitors, cholesterol absorption inhibitors, fibrates, niacin, statins, cholesteryl ester transfer protein inhibitors, bile acid sequestrants, anti-oxidants, vitamin E, probucol, IIb/IIIa antagonists, xemilofiban, and orbofiban.

43. The method of Embodiment 1 further comprising administering a third amount of an angiotensin converting enzyme inhibitor.

44. The method of Embodiment 43 wherein the aldosterone receptor antagonist is selected from the group consisting of eplerenone and spironolactone.

45. The method of Embodiment 43 wherein the aldosterone receptor antagonist is eplerenone.

46. The method of Embodiment 43 wherein the aldosterone receptor antagonist is spironolactone.

47. The method of Embodiment 43 wherein the anti-obesity agent is selected from the group consisting of orlistat and sibutramine and the pharmaceutically acceptable salts, esters, conjugate acids, and prodrugs thereof.

48. The method of Embodiment 43 wherein the angiotensin converting enzyme inhibitor is selected from the group consisting of benazapril, captopril, cilazapril, enalapril, fosinopril, lisinopril, perindopril, quinopril, ramipril, trandolapril, and the pharmaceutically acceptable salts, esters, conjugate acids, and prodrugs thereof.

49. The method of Embodiment 43, wherein the anti-obesity agent is selected from the group consisting of orlistat and sibutramine, and the pharmaceutically acceptable salts, esters, conjugate acids, and prodrugs thereof, and

wherein the angiotensin converting enzyme inhibitor is selected from the group consisting of benazapril, captopril, cilazapril, enalapril, fosinopril, lisinopril, perindopril, quinopril, ramipril, trandolapril, and the pharmaceutically acceptable salts, esters, conjugate acids, and prodrugs thereof.

50. The method of Embodiment 49 wherein the aldosterone receptor antagonist is eplerenone.

51. The method of Embodiment 49 wherein the aldosterone receptor antagonist is spironolactone.

52. The method of Embodiment 43 wherein the aldosterone receptor antagonist, anti-obesity agent, and angiotensin converting enzyme inhibitor are administered in a sequential manner.

53. The method of Embodiment 43 wherein the aldosterone receptor antagonist, anti-obesity agent, and angiotensin converting enzyme inhibitor are administered in a substantially simultaneous manner.

54. The method of Embodiment 43 wherein the aldosterone receptor antagonist is administered in a daily dose ranging from about 0.1 to about 2000 mg, the anti-obesity agent is administered in a daily dose ranging from about 0.1 to about 1000 mg, and the angiotensin converting enzyme inhibitor is administered in a daily dose ranging from about 0.1 to 1000 mg.

55. The method of Embodiment 43 wherein the first amount of the aldosterone receptor antagonist produces no substantial diuretic or anti-hypertensive effect in a subject.

56. A combination comprising an aldosterone receptor antagonist and a anti-obesity agent.

57. The combination of Embodiment 56 wherein the aldosterone receptor antagonist is eplerenone.

58. The combination of Embodiment 56 wherein the aldosterone receptor antagonist is spironolactone.

59. A pharmaceutical composition comprising a first amount of an aldosterone receptor antagonist, a second amount of an anti-obesity agent, and a pharmaceutically acceptable carrier.

60. The composition of Embodiment 59 wherein the first amount of the aldosterone receptor antagonist and the second amount of the anti-obesity agent together comprise a therapeutically-effective amount of the aldosterone receptor antagonist and anti-obesity agent.

61. The composition of Embodiment 59 wherein the aldosterone receptor antagonist is an epoxy-steroidal-type compound characterized in having a 9α-,11α-substituted epoxy moiety.

62. The composition of Embodiment 59 wherein the aldosterone receptor antagonist is eplerenone.

63. The composition of Embodiment 59 wherein the aldosterone receptor antagonist is a spirolactone-type compound.

64. The composition of Embodiment 59 wherein the aldosterone receptor antagonist is spironolactone.

65. The composition of Embodiment 59 wherein the aldosterone receptor antagonist is selected from the group consisting of:

pregn-4-ene-7,21-dicarboxylic acid, 9,11-epoxy-17-hydroxy-3-oxo,γ-lactone, methyl ester, (7α,11α,17α)-;

pregn-4-ene-7,21-dicarboxylic acid, 9,11-epoxy-17-hydroxy-3-oxo-dimethyl ester,(7α,11α,17α)-;

3′H-cyclopropa(6,7) pregna-4,6-diene-21-carboxylic acid, 9,11-epoxy-6,7-dihydro-17-hydroxy-3-oxo-,γ-lactone, (60,70,110,171)-;

pregn-4-ene-7,21-dicarboxylic acid, 9,11-epoxy-17-hydroxy-3-oxo-,7-(1-methylethyl) ester, monopotassium salt,(7α,11α,17α)-;

pregn-4-ene-7,21-dicarboxylic acid, 9,11,-epoxy-17-hydroxy-3-oxo-,7-methyl ester, monopotassium salt, (7α,1 (7α,11α,17α)-;

3′H-cyclopropa(6,7)pregna-1,4,6-triene-21-carboxylic acid, 9,11-epoxy-6,7-dihydro-17-hydroxy-3-oxo-, g-actone(6α,7α,11α)-;

3′H-cyclopropa(6,7)pregna-4,6-diene-21-carboxylic acid, 9,11-epoxy-6,7-dihydro-17-hydroxy-3-oxo-, methyl ester, (6α,7α,11α,17α)-;

3′H-cyclopropa(6,7)pregna-4,6-diene-21-carboxylic acid, 9,11-epoxy-6,7-dihydro-17-hydroxy-3-oxo-, monopotassium salt, (6α,7α,11α,17α)-;

3′H-cyclopropa(6,7)pregna-4,6-diene-21-carboxylic acid, 9,11-epoxy-6,7-dihydro-17-hydroxy-3-oxo-, g-lactone, (6α,7α,11α,17α)-;

pregn-4-ene-7,21-dicarboxylic acid, 9,11-epoxy-17-hydroxy-3-oxo-, γ-lactone, ethyl ester, (7α,11α,17α)-; and

pregn-4-ene-7,21-dicarboxylic acid, 9,11-epoxy-17-hydroxy-3-oxo-, γ-lactone, 1-methylethyl ester, (7α,11α,17α)-.

66. The composition of Embodiment 61 wherein the anti-obesity agent is selected from the group consisting of gastrointestinal lipase inhibitors, mixed norepinephrine and serotonin reuptake inhibitors, and the pharmaceutically acceptable salts, esters, conjugate acids, and prodrugs thereof.

67. The composition of Embodiment 66 wherein the aldosterone receptor antagonist is eplerenone.

68. The composition of Embodiment 59 wherein the anti-obesity agent is selected from the group consisting of orlistat and sibutramine and the pharmaceutically acceptable salts, esters, conjugate acids, and prodrugs thereof

69. The composition of Embodiment 68 wherein the aldosterone receptor antagonist is eplerenone.

70. The composition of Embodiment 59 wherein the anti-obesity agent is selected from the group consisting of a ciliary neurotrophic factor or one of its related variants or analogs; an appetite suppressant; a 5-HT-2c receptor agonist; a cannabinoid antagonist; a peptide or nonpeptide agonist of the cholecystokinin-A receptor; a peripherally acting agent that delays gastric emptying and increases satiety; an agonist of GLP-1 receptor; GLP-1s and related analogs, and the pharmaceutically acceptable salts, esters, conjugate acids, and prodrugs thereof.

71. The composition of Embodiment 70 wherein the aldosterone receptor antagonist is eplerenone.

72. The composition of Embodiment 59 wherein the anti-obesity agent is selected from the group consisting of Regeneron's Axokine, Phytopharm CP-644673/P-57, Biovitrum/GSK's BVT-933, Sanofi-Synthelabo's Rimonabant/SR-141716, GSK GI-181771, Aventis 1426, Exendin-4.

73. The composition of Embodiment 72 wherein the aldosterone receptor antagonist is eplerenone.

74. The composition of Embodiment 59 wherein the anti-obesity agent is selected from the group consisting of human growth hormone fragments; PTP-1B inhibitors; DPP-IV inhibitors, and the pharmaceutically acceptable salts, esters, conjugate acids, and prodrugs thereof.

75. The composition of Embodiment 74 wherein the aldosterone receptor antagonist is eplerenone.

76. The composition of Embodiment 59 wherein the anti-obesity agent is selected from the group consisting of neuropeptide Y1 antagonists; neuropeptide Y5 antagonists; thyroid hormone receptor beta agonists; glucocorticoid antagonists; melanocortin-4 receptor (MC-4) agonists; adiponectin/APM1/acrp30 and related analogs, and the pharmaceutically acceptable salts, esters, conjugate acids, and prodrugs thereof.

77. The composition of Embodiment 76 wherein the aldosterone receptor antagonist is eplerenone.

78. The composition of Embodiment 59 the anti-obesity agent is selected from the group consisting of 11-beta-hydroxysteroid dehydrogenase-1 inhibitors; fatty acid synthase inhibitors; acetyl CoA carboxylase inhibitors, and the pharmaceutically acceptable salts, esters, conjugate acids, and prodrugs thereof.

79. The composition of Embodiment 78 wherein the aldosterone receptor antagonist is eplerenone.

80. The composition of Embodiment 59 wherein the first amount of the aldosterone receptor antagonist produces no substantial diuretic or anti-hypertensive effect in a subject.

81. The composition of Embodiment 59 further comprising a third amount of a compound selected from the group consisting of renin inhibitors, angiotensin I antagonists, angiotensin II antagonists, angiotensin converting enzyme inhibitors, alpha-adrenergic receptor blockers, beta-adrenergic receptor blockers, calcium channel blockers, endothelin receptor antagonists, endothelin converting enzymes, vasodilators, diuretics, cyclooxygenase-2 inhibitors, apical sodium bile acid transport inhibitors, cholesterol absorption inhibitors, fibrates, niacin, statins, cholesteryl ester transfer protein inhibitors, bile acid sequestrants, anti-oxidants, vitamin E, probucol, IIb/IIIa antagonists, xemilofiban, and orbofiban.

82. The composition of Embodiment 59 further comprising administering a third amount of an angiotensin converting enzyme inhibitor.

83. The composition of Embodiment 82 wherein the aldosterone receptor antagonist is selected from the group consisting of eplerenone and spironolactone.

84. The composition of Embodiment 82 wherein the aldosterone receptor antagonist is eplerenone.

85. The composition of Embodiment 82 wherein the aldosterone receptor antagonist is spironolactone.

86. The composition of Embodiment 82 wherein the anti-obesity agent is selected from the group consisting of orlistat and sibutramine, and the pharmaceutically acceptable salts, esters, conjugate acids, and prodrugs thereof.

87. The composition of Embodiment 82 wherein the angiotensin converting enzyme inhibitor is selected from the group consisting of benazapril, captopril, cilazapril, enalapril, fosinopril, lisinopril, perindopril, quinopril, ramipril, trandolapril, and the pharmaceutically acceptable salts, esters, conjugate acids, and prodrugs thereof.

88. The composition of Embodiment 84, wherein the anti-obesity agent is selected from the group consisting of orlistat and sibutramine, and the pharmaceutically acceptable salts, esters, conjugate acids, and prodrugs thereof, and

wherein the angiotensin converting enzyme inhibitor is selected from the group consisting of benazapril, captopril, cilazapril, enalapril, fosinopril, lisinopril, perindopril, quinopril, ramipril, trandolapril, and the pharmaceutically acceptable salts, esters, conjugate acids, and prodrugs thereof.

89. The composition of Embodiment 88 wherein the aldosterone receptor antagonist is eplerenone.

90. The composition of Embodiment 88 wherein the aldosterone receptor antagonist is spironolactone.

91. A kit containing a first amount of an aldosterone receptor antagonist and a second amount of an anti-obesity agent.

92. The kit of Embodiment 91 comprising the first amount of the aldosterone receptor antagonist in a unit dosage form, and the second amount of an anti-obesity agent in a unit dosage form.

93. The kit of Embodiment 91 wherein the aldosterone receptor antagonist is an epoxy-steroidal-type compound characterized in having a 9α-,11α-substituted epoxy moiety.

94. The kit of Embodiment 91 wherein the aldosterone receptor antagonist is eplerenone.

95. The kit of Embodiment 91 wherein the aldosterone receptor antagonist is a spirolactone-type compound.

96. The kit of Embodiment 91 wherein the aldosterone receptor antagonist is spironolactone.

97. The kit of Embodiment 91 wherein the aldosterone receptor antagonist is selected from the group consisting of:

pregn-4-ene-7,21-dicarboxylic acid, 9,11-epoxy-17-hydroxy-3-oxo,γ-lactone, methyl ester, (7α,11α,17α)-;

pregn-4-ene-7,21-dicarboxylic acid, 9,11-epoxy-17-hydroxy-3-oxo-dimethyl ester,(7α,11α,17α)-;

3′H-cyclopropa(6,7) pregna-4,6-diene-21-carboxylic acid, 9,11-epoxy-6,7-dihydro-17-hydroxy-3-oxo-,γ-lactone, (6β,7β,11β,17β)-;

pregn-4-ene-7,21-dicarboxylic acid, 9,11-epoxy-17-hydroxy-3-oxo-,7-(1-methylethyl) ester, monopotassium salt,(7α,11α,17α)-;

pregn-4-ene-7,21-dicarboxylic acid, 9,11,-epoxy-17-hydroxy-3-oxo-,7-methyl ester, monopotassium salt, (7α,11α,17α)-;

3′H-cyclopropa(6,7)pregna-1,4,6-triene-21-carboxylic acid, 9,11-epoxy-6,7-dihydro-17-hydroxy-3-oxo-γ-actone(6α,7α,11α)-;

3′H-cyclopropa(6,7)pregna-4,6-diene-21-carboxylic acid, 9,11-epoxy-6,7-dihydro-17-hydroxy-3-oxo-, methyl ester, (6α,7α,11α,17α)-;

3′H-cyclopropa(6,7)pregna-4,6-diene-21-carboxylic acid, 9,11-epoxy-6,7-dihydro-17-hydroxy-3-oxo-, monopotassium salt, (6α,7α,11α,17α)-;

3′H-cyclopropa(6,7)pregna-4,6-diene-21-carboxylic acid, 9,11-epoxy-6,7-dihydro-17-hydroxy-3-oxo-, γ-lactone, (6α,7α,11α,17α)-;

pregn-4-ene-7,21-dicarboxylic acid, 9,11-epoxy-17-hydroxy-3-oxo-,γ-lactone, ethyl ester, (7α,11α,17α)-; and

pregn-4-ene-7,21-dicarboxylic acid, 9,11-epoxy-17-hydroxy-3-oxo-,γ-lactone, 1-methylethyl ester, (7α,11α,17α)-.

98. The kit of Embodiment 91 wherein the anti-obesity agent is selected from the group consisting of gastrointestinal lipase inhibitors, mixed norepinephrine and serotonin reuptake inhibitors, and the pharmaceutically acceptable salts, esters, conjugate acids, and prodrugs thereof.

99. The kit of Embodiment 98 wherein the aldosterone receptor antagonist is eplerenone.

100. The kit of Embodiment 91 wherein the anti-obesity agent is selected from the group consisting of orlistat and sibutramine and the pharmaceutically acceptable salts, esters, conjugate acids, and prodrugs thereof.

101. The kit of Embodiment 100 wherein the aldosterone receptor antagonist is eplerenone.

102. The kit of Embodiment 91 wherein the anti-obesity agent is selected from the group consisting of a ciliary neurotrophic factor or one of its related variants or analogs; an appetite suppressant; a 5-HT-2c receptor agonist; a cannabinoid antagonist; a peptide or nonpeptide agonist of the cholecystokinin-A receptor; a peripherally acting agent that delays gastric emptying and increases satiety; an agonist of GLP-1 receptor: GLP-1s and related analogs, and the pharmaceutically acceptable salts, esters, conjugate acids, and prodrugs thereof.

103. The kit of Embodiment 102 wherein the aldosterone receptor antagonist is eplerenone.

104. The kit of Embodiment 91 wherein the anti-obesity agent is selected from the group consisting of Regeneron's Axokine, Pfizer/Phytopharm CP-644673/P-57, Biovitrum/GSK's BVT-933, Sanofi-Synthelabo's Rimonabant/SR-141716, GSK GI-181771, Aventis 1426, Exendin-4.

105. The kit of Embodiment 104 wherein the aldosterone receptor antagonist is eplerenone.

106. The kit of Embodiment 91 wherein the anti-obesity agent is selected from the group consisting of human growth hormone fragments; PTP-1B inhibitors; DPP-IV inhibitors, and the pharmaceutically acceptable salts, esters, conjugate acids, and prodrugs thereof.

107. The kit of Embodiment 106 wherein the aldosterone receptor antagonist is eplerenone.

108. The kit of Embodiment 91 wherein the anti-obesity agent is selected from the group consisting of neuropeptide Y1 antagonists; neuropeptide Y5 antagonists; thyroid hormone receptor beta agonists; glucocorticoid antagonists; melanocortin-4 receptor (MC-4) agonists; adiponectin/APM1/acrp30 and related analogs, and the pharmaceutically acceptable salts, esters, conjugate acids, and prodrugs thereof.

109. The kit of Embodiment 108 wherein the aldosterone receptor antagonist is eplerenone.

110. The kit of Embodiment 91 the anti-obesity agent is selected from the group consisting of 11-beta-hydroxysteroid dehydrogenase-1 inhibitors; fatty acid synthase inhibitors; acetyl CoA carboxylase inhibitors, and the pharmaceutically acceptable salts, esters, conjugate acids, and prodrugs thereof.

111. The kit of Embodiment 110 wherein the aldosterone receptor antagonist is eplerenone.

112. The kit of Embodiment 91 further comprising a third amount of an angiotensin converting enzyme inhibitor.

113. The kit of Embodiment 112 wherein the aldosterone receptor antagonist is selected from the group consisting of eplerenone and spironolactone.

114. The kit of Embodiment 112 wherein the aldosterone receptor antagonist is spironolactone.

115. The kit of Embodiment 112 wherein the aldosterone receptor antagonist is eplerenone.

116. The kit of Embodiment 112 wherein the anti-obesity agent is selected from the group consisting of orlistat and sibutramine and the pharmaceutically acceptable salts, esters, conjugate acids, and prodrugs thereof.

117. The kit of Embodiment 112 wherein the angiotensin converting enzyme inhibitor is selected from the group consisting of benazapril, captopril, cilazapril, enalapril, fosinopril, lisinopril, perindopril, quinopril, ramipril, trandolapril, and the pharmaceutically acceptable salts, esters, conjugate acids, and prodrugs thereof.

118. The kit of Embodiment 112 wherein the anti-obesity agent is selected from the group consisting of orlistat and sibutramine, and the pharmaceutically acceptable salts, esters, conjugate acids, and prodrugs thereof, and

wherein the angiotensin converting enzyme inhibitor is selected from the group consisting of benazapril, captopril, cilazapril, enalapril, fosinopril, lisinopril, perindopril, quinopril, ramipril, trandolapril, and the pharmaceutically acceptable salts, esters, conjugate acids, and prodrugs thereof.

119. The kit of Embodiment 118 wherein the aldosterone receptor antagonist is eplerenone.

120. The kit of Embodiment 118 wherein the aldosterone receptor antagonist is spironolactone.

Further Additional Embodiments

1′. A method for the treatment of a cardiovascular-related condition, the method comprising administering to a subject susceptible to or afflicted with such condition a first amount of an aldosterone receptor antagonist and a second amount of an anti-obesity agent, wherein the first amount of the aldosterone receptor antagonist and the second amount of the anti-obesity agent together comprise a therapeutically-effective amount of the aldosterone receptor antagonist and anti-obesity agent.

2′. The method of Embodiment 1′ wherein the aldosterone receptor antagonist is eplerenone.

3′. The method of Embodiment 2′ wherein the eplerenone is administered in a daily dose range from about 1 mg to about 250 mg.

4′. The method of Embodiment 2′ wherein the cardiovascular-related condition is selected from the group consisting of coronary artery disease, hypertension, cardiovascular disease, renal dysfunction, liver disease, heart failure, cerebrovascular disease, vascular disease, retinopathy, neuropathy, hyperglycemia, hyperinsulinemia, insulin resistance, edema, endothelial dysfunction, baroreceptor dysfunction, of heart failure, arrhythmia, diastolic dysfunction, systolic dysfunction, ischemia, hypertrophic cardiomyopathy, sudden cardiac death, myocardial fibrosis, vascular fibrosis, impaired arterial compliance, myocardial necrotic lesions, vascular damage, myocardial infarction, left ventricular hypertrophy, decreased ejection fraction, cardiac lesions, vascular wall hypertrophy, endothelial thickening, endothelial dysfunction and fibrinoid necrosis of coronary arteries.

5′. The method of Embodiment 4′ wherein the cardiovascular-related condition is hypertension.

6′. The method of Embodiment 4′ wherein the cardiovascular-related condition is renal dysfunction which is selected from the group consisting of glomerulosclerosis, end-stage renal disease, diabetic nephropathy, reduced renal blood flow, increased glomerular filtration fraction, proteinuria, decreased glomerular filtration rate, decreased creatinine clearance, microalbuminuria, renal arteriopathy, ischemic lesions, thrombotic lesions, global fibrinoid necrosis, focal thrombosis of glomerular capillaries, swelling and proliferation of intracapillary cells, swelling and proliferation of extracapillary cells, expansion of reticulated mesangial matrix with or without significant hypercellularity, and malignant nephrosclerosis.

7′. The method of Embodiment 4′ wherein the cardiovascular-related condition is heart failure.

8′. The method of embodiment 4′ wherein the cardiovascular related condition is endothelial dysfunction.

9′. The method of embodiment 4′ wherein the cardiovascular related condition is vascular disease.

10′. The method of embodiment 4′ wherein the cardiovascular related condition is cerebrovascular disease.

11′. The method of embodiment 10′ wherein the cerebrovascular disease is stroke.

12′. The method of embodiment 9′ wherein the vascular disease is selected from the group consisting of thrombotic vascular disease, proliferative arteriopathy, atherosclerosis, and decreased vascular compliance.

13′. The method of embodiment 4′ wherein the cardiovascular related condition is edema.

14′. The method of embodiment 13′ wherein the edema is selected from the group consisting of peripheral tissue edema, hepatic congestion, splenic congestion, liver ascites, respiratory congestion, and lung congestion.

15′. The method of embodiment 4′ wherein the cardiovascular related condition is selected from the group consisting of hyperglycemia, hyperinsulinemia, and insulin resistance.

16′. The method of embodiment 15′ wherein the cardiovascular related condition is selected from the group consisting of Type I diabetes mellitus, Type II diabetes mellitus, insulin resistance, pre-diabetic state, and metabolic syndrome.

17′. The method of embodiment 4′ wherein the cardiovascular related condition is selected from the group consisting of coronary heart disease, hypertension, cardiovascular disease, stroke, and Type II diabetes mellitus.

18′. The method of embodiment 17′ wherein the cardiovascular related condition is selected from the group consisting of coronary heart disease, hypertension, heart failure, left ventricular hypertrophy and stroke.

19′. The method of embodiment 1′ wherein the aldosterone receptor antagonist is selected from the group consisting of:

pregn-4-ene-7,21-dicarboxylic acid, 9,11-epoxy-17-hydroxy-3-oxo,γ-lactone, methyl ester, (7α,11α,17α)-;

pregn-4-ene-7,21-dicarboxylic acid, 9,11-epoxy-17-hydroxy-3-oxo-dimethyl ester,(7α,11α,17α)-;

3′H-cyclopropa(6,7) pregna-4,6-diene-21-carboxylic acid, 9,11-epoxy-6,7-dihydro-17-hydroxy-3-oxo-,γ-lactone, (6β,7β, 11β,17α)β-;

pregn-4-ene-7,21-dicarboxylic acid, 9,11-epoxy-17-hydroxy-3-oxo-,7-(1-methylethyl) ester, monopotassium salt,(7α,11α,17α)-;

pregn-4-ene-7,21-dicarboxylic acid, 9,11,-epoxy-17-hydroxy-3-oxo-,7-methyl ester, monopotassium salt, (7α,11α,17α)-;

3′H-cyclopropa(6,7)pregna-1,4,6-triene-21-carboxylic acid, 9,11-epoxy-6,7-dihydro-17-hydroxy-3-oxo-γ-actone(6α,7α,11α)-;

3′H-cyclopropa(6,7)pregna-4,6-diene-21-carboxylic acid, 9,11-epoxy-6,7-dihydro-17-hydroxy-3-oxo-, methyl ester, (6α,7α,11α,17α)-;

3′H-cyclopropa(6,7)pregna-4,6-diene-21-carboxylic acid, 9,11-epoxy-6,7-dihydro-17-hydroxy-3-oxo-, monopotassium salt, (6α,7α,11α,17α)-;

3′H-cyclopropa(6,7)pregna-4,6-diene-21-carboxylic acid, 9,11-epoxy-6,7-dihydro-17-hydroxy-3-oxo-, γ-lactone, (6α,7α,11α,17α)-;

pregn-4-ene-7,21-dicarboxylic acid, 9,11-epoxy-17-hydroxy-3-oxo-,γ-lactone, ethyl ester, (7α,11α,17α)-; and

pregn-4-ene-7,21-dicarboxylic acid, 9,11-epoxy-17-hydroxy-3-oxo-,γ-lactone, 1-methylethyl ester, (7α,11α,17α)-.

20′. The method of Embodiment 1′ wherein the anti-obesity agent is selected from the group consisting of gastrointestinal lipase inhibitors, mixed norepinephrine and serotonin reuptake inhibitors, and the pharmaceutically acceptable salts, esters, conjugate acids, and prodrugs thereof.

21′. The method of embodiment 20′ wherein the aldosterone receptor antagonist is eplerenone.

22′. The method of Embodiment 2′ wherein the anti-obesity agent is selected from the group consisting of orlistat, sibutramine, and pharmaceutically acceptable salts, esters, conjugate acids, and prodrugs thereof.

23′. The method of Embodiment 21′ wherein the anti-obesity agent is orlistat.

24′. The method of Embodiment 21′ wherein the anti-obesity agent is sibutramine.

25′. The method of Embodiment 1′ wherein the aldosterone receptor antagonist is spironolactone.

26′. The method of Embodiment 1′ wherein the anti-obesity agent is orlistat.

27′. The method of Embodiment 1′ wherein the anti-obesity agent is sibutramine.

28′. The method of Embodiment 1′ wherein the anti-obesity agent is selected from the group consisting of a ciliary neurotrophic factor or one of its related variants or analogs; an appetite suppressant; a 5-HT-2c receptor agonist; a cannabinoid antagonist; a peptide or nonpeptide agonist of the cholecystokinin-A receptor; a peripherally acting agent that delays gastric emptying and increases satiety; an agonist of GLP-1 receptor; GLP-1s and related analogs; and the pharmaceutically acceptable salts, esters, conjugate acids, and prodrugs thereof.

29′. The method of Embodiment 28′ wherein the aldosterone receptor antagonist is eplerenone.

30′. The method of Embodiment 1′ wherein the anti-obesity agent is selected from the group consisting of Axokine, CP-644673/P-57, BVT-933, Rimonabant/SR-141716, GI-181771, HMR-1426, and Exendin-4.

31′. The method of Embodiment 30′ wherein the aldosterone receptor antagonist is eplerenone.

32′. The method of Embodiment 1′ wherein the anti-obesity agent is selected from the group consisting of human growth hormone fragments; PTP-1B inhibitors; DPP-IV inhibitors; and the pharmaceutically acceptable salts, esters, conjugate acids, and prodrugs thereof.

33′. The method of Embodiment 32′ wherein the aldosterone receptor antagonist is eplerenone.

34′. The method of Embodiment 1′ wherein the anti-obesity agent is selected from the group consisting of neuropeptide Y1 antagonists; neuropeptide Y5 antagonists; thyroid hormone receptor beta agonists; glucocorticoid antagonists; melanocortin-4 receptor (MC-4) agonists; adiponectin/APM1/acrp30 and related analogs; and the pharmaceutically acceptable salts, esters, conjugate acids, and prodrugs thereof.

35′. The method of Embodiment 34″ wherein the aldosterone receptor antagonist is eplerenone.

36′. The method of Embodiment 1′ the anti-obesity agent is selected from the group consisting of 11-beta-hydroxysteroid dehydrogenase-1 inhibitors; fatty acid synthase inhibitors; acetyl CoA carboxylase inhibitors, and the pharmaceutically acceptable salts, esters, conjugate acids, and prodrugs thereof.

37′. The method of Embodiment 36″ wherein the aldosterone receptor antagonist is eplerenone.

38′. The method of Embodiment 1′ wherein the aldosterone receptor antagonist and the anti-obesity agent are administered in a sequential manner.

39′. The method of Embodiment 1′ wherein the aldosterone receptor antagonist and the neutral endopeptidase inhibitor are administered in a substantially simultaneous manner.

40′. The method of Embodiment 1′ wherein the aldosterone receptor antagonist is administered in a daily dose ranging from about 0.1 to about 2000 mg, and the anti-obesity agent is administered in a daily dose ranging from about 0.1 to about 1000 mg.

41′. The method of embodiment 40′ wherein the aldosterone receptor antagonist is eplerenone.

42′. The method of embodiment 41′ wherein the eplerenone is provided in a daily dose ranging from about 1 to about 250 mg.

43′. The method of Embodiment 1′ wherein the first amount of the aldosterone receptor antagonist produces no substantial diuretic or anti-hypertensive effect in a subject.

44′. The method of Embodiment 1′ further comprising administering a third amount of a compound selected from the group consisting of anti-diabetic agents, renin inhibitors, angiotensin I antagonists, angiotensin II antagonists, angiotensin converting enzyme inhibitors, alpha-adrenergic receptor blockers, beta-adrenergic receptor blockers, calcium channel blockers, endothelin receptor antagonists, endothelin converting enzymes, vasodilators, diuretics, cyclooxygenase-2 inhibitors, apical sodium bile acid transport inhibitors, cholesterol absorption inhibitors, fibrates, niacin, statins, cholesteryl ester transfer protein inhibitors, bile acid sequestrants, anti-oxidants, vitamin E, probucol, IIb/IIIa antagonists, xemilofiban, and orbofiban.

45′. A pharmaceutical composition comprising a first amount of an aldosterone receptor antagonist, a second amount of an anti-obesity agent, and a pharmaceutically acceptable carrier, wherein the first amount of the aldosterone receptor antagonist and the second amount of the anti-obesity agent together comprise a therapeutically-effective amount of the aldosterone receptor antagonist and anti-obesity agent.

46′. The composition of Embodiment 45′ wherein the aldosterone receptor antagonist is selected from the group consisting of:

pregn-4-ene-7,21-dicarboxylic acid, 9,11-epoxy-17-hydroxy-3-oxo,γ-lactone, methyl ester, (7α,11α,17α)-;

pregn-4-ene-7,21-dicarboxylic acid, 9,11-epoxy-17-hydroxy-3-oxo-dimethyl ester,(7α,11α,17α)-;

3′H-cyclopropa(6,7) pregna-4,6-diene-21-carboxylic acid, 9,11-epoxy-6,7-dihydro-17-hydroxy-3-oxo-γ-lactone, (6β,7β,11β,17α)-;

pregn-4-ene-7,21-dicarboxylic acid, 9,11-epoxy-17-hydroxy-3-oxo-,7-(1-methylethyl) ester, monopotassium salt,(7α,11α,17α)-;

pregn-4-ene-7,21-dicarboxylic acid, 9,11,-epoxy-17-hydroxy-3-oxo-,7-methyl ester, monopotassium salt, (7α,11α,17α)-;

3′H-cyclopropa(6,7)pregna-1,4,6-triene-21-carboxylic acid, 9,11-epoxy-6,7-dihydro-17-hydroxy-3-oxo-, g-actone(6α,7α,11α)-;

3′H-cyclopropa(6,7)pregna-4,6-diene-21-carboxylic acid, 9,11-epoxy-6,7-dihydro-17-hydroxy-3-oxo-, methyl ester, (6α,7α,11α,17α)-;

3′H-cyclopropa(6,7)pregna-4,6-diene-21-carboxylic acid, 9,11-epoxy-6,7-dihydro-17-hydroxy-3-oxo-, monopotassium salt, (7α,11α,17α)-;

3′H-cyclopropa(6,7)pregna-4,6-diene-21-carboxylic acid, 9,11-epoxy-6,7-dihydro-17-hydroxy-3-oxo-, g-lactone, (6α,7α,11α,17α)-;

pregn-4-ene-7,21-dicarboxylic acid, 9,11-epoxy-17-hydroxy-3-oxo-, g-lactone, ethyl ester, (7α,11α,17α)-; and

pregn-4-ene-7,21-dicarboxylic acid, 9,11-epoxy-17-hydroxy-3-oxo-, g-lactone, 1-methylethyl ester, (7α,11α,17α)-.

47′. The composition of Embodiment 45′ wherein the aldosterone receptor antagonist is eplerenone.

48′. The composition of embodiment 47′ wherein the eplerenone is administered in a daily dose range from about 1 to about 250 mg.

49′. The composition of Embodiment 45′ wherein the anti-obesity agent is selected from the group consisting of gastrointestinal lipase inhibitors, mixed norepinephrine and serotonin reuptake inhibitors, and the pharmaceutically acceptable salts, esters, conjugate acids, and prodrugs thereof.

50′. The composition of Embodiment 49′ wherein the aldosterone receptor antagonist is eplerenone.

51′. The composition of Embodiment 45′ wherein the anti-obesity agent is selected from the group consisting of orlistat and sibutramine and the pharmaceutically acceptable salts, esters, conjugate acids, and prodrugs thereof.

52′. The composition of Embodiment 51′ wherein the aldosterone receptor antagonist is eplerenone.

53′. The composition of Embodiment 45′ wherein the anti-obesity agent is selected from the group consisting of a ciliary neurotrophic factor or one of its related variants or analogs; an appetite suppressant; a 5-HT-2c receptor agonist; a cannabinoid antagonist; a peptide or nonpeptide agonist of the cholecystokinin-A receptor; a peripherally acting agent that delays gastric emptying and increases satiety; an agonist of GLP-1 receptor; GLP-1s and related analogs, and the pharmaceutically acceptable salts, esters, conjugate acids, and prodrugs thereof.

54′. The composition of Embodiment 53′ wherein the aldosterone receptor antagonist is eplerenone.

55′. The composition of Embodiment 45′ wherein the anti-obesity agent is selected from the group consisting of Axokine, CP-644673/P-57, BVT-933, Rimonabant/SR-141716, GI-181771, HMR-1426, and Exendin-4.

56′. The composition of Embodiment 55′ wherein the aldosterone receptor antagonist is eplerenone.

57′. The composition of Embodiment 45′ wherein the anti-obesity agent is selected from the group consisting of human growth hormone fragments; PTP-1B inhibitors; DPP-IV inhibitors; and the pharmaceutically acceptable salts, esters, conjugate acids, and prodrugs thereof.

58′. The composition of Embodiment 57′ wherein the aldosterone receptor antagonist is eplerenone.

59′. The composition of Embodiment 45′ wherein the anti-obesity agent is selected from the group consisting of neuropeptide Y1 antagonists; neuropeptide Y5 antagonists; thyroid hormone receptor beta agonists; glucocorticoid antagonists; melanocortin-4 receptor (MC-4) agonists; adiponectin/APM1/acrp30 and related analogs, and the pharmaceutically acceptable salts, esters, conjugate acids, and prodrugs thereof.

60′. The composition of Embodiment 59′ wherein the aldosterone receptor antagonist is eplerenone.

61′. The composition of Embodiment 45′ the anti-obesity agent is selected from the group consisting of 11-beta-hydroxysteroid dehydrogenase-1 dehydrogenase-1 inhibitors; fatty acid synthase inhibitors; acetyl CoA carboxylase inhibitors; and the pharmaceutically acceptable salts, esters, conjugate acids, and prodrugs thereof.

62′. The composition of Embodiment 61′ wherein the aldosterone receptor antagonist is eplerenone.

63′. The composition of Embodiment 45′ wherein the first amount of the aldosterone receptor antagonist produces no substantial diuretic or anti-hypertensive effect in a subject.

64′. The composition of Embodiment 45′ further comprising a third amount of a compound selected from the group consisting of anti-diabetic agents, renin inhibitors, angiotensin I antagonists, angiotensin II antagonists, angiotensin converting enzyme inhibitors, alpha-adrenergic receptor blockers, beta-adrenergic receptor blockers, calcium channel blockers, endothelin receptor antagonists, endothelin converting enzymes, vasodilators, diuretics, cyclooxygenase-2 inhibitors, apical sodium bile acid transport inhibitors, cholesterol absorption inhibitors, fibrates, niacin, statins, cholesteryl ester transfer protein inhibitors, bile acid sequestrants, anti-oxidants, vitamin E, probucol, IIb/IIIa antagonists, xemilofiban, and orbofiban.

65′. A kit containing a first amount of an aldosterone receptor antagonist and a second amount of an anti-obesity agent.

66′. The kit of Embodiment 65′ comprising the first amount of the aldosterone receptor antagonist in a unit dosage form, and the second amount of an anti-obesity agent in a unit dosage form.

67′. The kit of Embodiment 65′ wherein the aldosterone receptor antagonist is eplerenone.

68′. The kit of Embodiment 65′ wherein the anti-obesity agent is selected from the group consisting of gastrointestinal lipase inhibitors, mixed norepinephrine and serotonin reuptake inhibitors, and the pharmaceutically acceptable salts, esters, conjugate acids, and prodrugs thereof.

69′. The kit of Embodiment 68′ wherein the aldosterone receptor antagonist is eplerenone.

70′. The kit of Embodiment 65′ wherein the anti-obesity agent is selected from the group consisting of orlistat and sibutramine and the pharmaceutically acceptable salts, esters, conjugate acids, and prodrugs thereof.

71′. The kit of Embodiment 70′ wherein the aldosterone receptor antagonist is eplerenone.

72′. The kit of Embodiment 65′ wherein the anti-obesity agent is selected from the group consisting of a ciliary neurotrophic factor or one of its related variants or analogs; an appetite suppressant; a 5-HT-2c receptor agonist; a cannabinoid antagonist; a peptide or nonpeptide agonist of the cholecystokinin-A receptor; a peripherally acting agent that delays gastric emptying and increases satiety; an agonist of GLP-1 receptor; GLP-1s and related analogs; and the pharmaceutically acceptable salts, esters, conjugate acids, and prodrugs thereof.

73′. The kit of Embodiment 72′ wherein the aldosterone receptor antagonist is eplerenone.

74′. The kit of Embodiment 65′ wherein the anti-obesity agent is selected from the group consisting of Axokine, CP-644673/P-57, BVT-933, Rimonabant/SR-141716, GI-181771, HMR-1426, and Exendin-4.

75′. The kit of Embodiment 74′ wherein the aldosterone receptor antagonist is eplerenone.

76′. The kit of Embodiment 65′ wherein the anti-obesity agent is selected from the group consisting of human growth hormone fragments; PTP-1B inhibitors; DPP-IV inhibitors; and the pharmaceutically acceptable salts, esters, conjugate acids, and prodrugs thereof.

77′. The kit of Embodiment 76′ wherein the aldosterone receptor antagonist is eplerenone.

78′. The kit of Embodiment 65′ wherein the anti-obesity agent is selected from the group consisting of neuropeptide Y1 antagonists; neuropeptide Y5 antagonists; thyroid hormone receptor beta agonists; glucocorticoid antagonists; melanocortin-4 receptor (MC-4) agonists; adiponectin/APM1/acrp30 and related analogs; and the pharmaceutically acceptable salts, esters, conjugate acids, and prodrugs thereof.

79′. The kit of Embodiment 78′ wherein the aldosterone receptor antagonist is eplerenone.

80′. The kit of Embodiment 65′ the anti-obesity agent is selected from the group consisting of 11-beta-hydroxysteroid dehydrogenase-1 inhibitors; fatty acid synthase inhibitors; acetyl CoA carboxylase inhibitors; and the pharmaceutically acceptable salts, esters, conjugate acids, and prodrugs thereof.

81′. The kit of Embodiment 80′ wherein the aldosterone receptor antagonist is eplerenone.

82′. The kit of Embodiment 65′ wherein the aldosterone receptor antagonist is spironolactone.

83′. The kit of Embodiment 65′ wherein the anti-obesity agent is selected from the group consisting of orlistat and sibutramine and the pharmaceutically acceptable salts, esters, conjugate acids, and prodrugs thereof.

84′. The method of Embodiment 44′ wherein the angiotensin converting enzyme inhibitor is selected from the group consisting of benazapril; captopril; cilazapril; enalapril; fosinopril; lisinopril; perindopril; quinopril; ramipril; trandolapril; and the pharmaceutically acceptable salts, esters, conjugate acids, and prodrugs thereof.

85′. The method of Embodiment 84′ wherein the anti-obesity agent is selected from the group consisting of orlistat and sibutramine, and the pharmaceutically acceptable salts, esters, conjugate acids, and prodrugs thereof.

86′. The method of Embodiment 85′ wherein the aldosterone receptor antagonist is eplerenone.

87′. The method of Embodiment 44′ wherein the anti-diabetic agent is metformin.

88′. The method of Embodiment 1′ wherein the subject is a human.

89′. The method of Embodiment 88′ wherein the subject is obese.

90′. The method of Embodiment 89′ wherein the subject is hypertensive.

91′. The method of Embodiment 90′ wherein the human has undergone unilateral nephrectomy.

92′. The method of Embodiment 1′ wherein the subject is a human having undergone unilateral nephrectomy.

93′. The method of Embodiment 92′ wherein the human is obese.

94′. The method of Embodiment 92′ wherein the human is hypertensive.

All citations to references, books, magazines, journal articles, patents, patent applications or any other publications, etc., recited in this application are expressly incorporated herein by reference.

What is claimed is:

1. A method for the treatment of a cardiovascular-related condition, the method comprising administering to a subject susceptible to or afflicted with such condition a first amount of an aldosterone receptor antagonist and a second amount of an anti-obesity agent, wherein the first amount of the aldosterone receptor antagonist and the second amount of the anti-obesity agent together comprise a therapeutically-effective amount of the aldosterone receptor antagonist and anti-obesity agent.

2. The method of claim 1 wherein the aldosterone receptor antagonist is eplerenone.

3. The method of claim 2 wherein the eplerenone is administered in a daily dose range from about 1 mg to about 250 mg.

4. The method of claim 2 wherein the cardiovascular-related condition is selected from the group consisting of coronary artery disease, hypertension, cardiovascular disease, renal dysfunction, liver disease, heart failure, cerebrovascular disease, vascular disease, retinopathy, neuropathy, hyperglycemia, hyperinsulinemia, insulin resistance, edema, endothelial dysfunction, baroreceptor dysfunction, of heart failure, arrhythmia, diastolic dysfunction, systolic dysfunction, ischemia, hypertrophic cardiomyopathy, sudden cardiac death, myocardial fibrosis, vascular fibrosis, impaired arterial compliance, myocardial necrotic lesions, vascular damage, myocardial infarction, left ventricular hypertrophy, decreased ejection fraction, cardiac lesions, vascular wall hypertrophy, endothelial thickening, endothelial dysfunction and fibrinoid necrosis of coronary arteries.

5. The method of claim 4 wherein the cardiovascular-related condition is hypertension.

6. The method of claim 4 wherein the cardiovascular-related condition is renal dysfunction which is selected from the group consisting of glomerulosclerosis, end-stage renal disease, diabetic nephropathy, reduced renal blood flow, increased glomerular filtration fraction, proteinuria, decreased glomerular filtration rate, decreased creatinine clearance, microalbuminuria, renal arteriopathy, ischemic lesions, thrombotic lesions, global fibrinoid necrosis, focal thrombosis of glomerular capillaries, swelling and proliferation of intracapillary cells, swelling and proliferation of extracapillary cells, expansion of reticulated mesangial matrix with or without significant hypercellularity, and malignant nephrosclerosis.

7. The method of claim 4 wherein the cardiovascular-related condition is heart failure.

8. The method of claim 4 wherein the cardiovascular related condition is endothelial dysfunction.

9. The method of claim 4 wherein the cardiovascular related condition is vascular disease.

10. The method of claim 4 wherein the cardiovascular related condition is cerebrovascular disease.

11. The method of claim 10 wherein the cerebrovascular disease is stroke.

12. The method of claim 9 wherein the vascular disease is selected from the group consisting of thrombotic vascular disease, proliferative arteriopathy, atherosclerosis, and decreased vascular compliance.

13. The method of claim 4 wherein the cardiovascular related condition is edema.

14. The method of claim 13 wherein the edema is selected from the group consisting of peripheral tissue edema, hepatic congestion, splenic congestion, liver ascites, respiratory congestion, and lung congestion.

15. The method of claim 4 wherein the cardiovascular related condition is selected from the group consisting of hyperglycemia, hyperinsulinemia, and insulin resistance.

16. The method of claim 15 wherein the cardiovascular related condition is selected from the group consisting of Type I diabetes mellitus, Type II diabetes mellitus, insulin resistance, pre-diabetic state, and metabolic syndrome.

17. The method of claim 4 wherein the cardiovascular related condition is selected from the group consisting of coronary heart disease, hypertension, cardiovascular disease, stroke, and Type II diabetes mellitus.

18. The method of claim 17 wherein the cardiovascular related condition is selected from the group consisting of coronary heart disease, hypertension, heart failure, left ventricular hypertrophy and stroke.

19. The method of claim 1 wherein the aldosterone receptor antagonist is selected from the group consisting of:
pregn-4-ene-7,21-dicarboxylic acid, 9,11-epoxy-17-hydroxy-3-oxo,γ-lactone, methyl ester, (7α,11α,17α)-;
pregn-4-ene-7,21-dicarboxylic acid, 9,11-epoxy-17-hydroxy-3-oxo-dimethyl ester,(7α,11α,17α)-;
3′H-cyclopropa(6,7) pregna-4,6-diene-21-carboxylic acid, 9,11-epoxy-6,7-dihydro-17-hydroxy-3-oxo-,γ-lactone, (60,70,110,170)-;
pregn-4-ene-7,21-dicarboxylic acid, 9,11-epoxy-17-hydroxy-3-oxo-,7-(1-methylethyl) ester, monopotassium salt,(7α,11α,17α)-;
pregn-4-ene-7,21-dicarboxylic acid, 9,11,-epoxy-17-hydroxy-3-oxo-,7-methyl ester, monopotassium salt, (7α,11α,17α)-;
3′H-cyclopropa(6,7)pregna-1,4,6-triene-21-carboxylic acid, 9,11-epoxy-6,7-dihydro-17-hydroxy-3-oxo-,γ-actone(6α,7α,111α)-;
3′H-cyclopropa(6,7)pregna-4,6-diene-21-carboxylic acid, 9,11-epoxy-6,7-dihydro-17-hydroxy-3-oxo-, methyl ester, (7α,11α,17α)-;
3′H-cyclopropa(6,7)pregna-4,6-diene-21-carboxylic acid, 9,11-epoxy-6,7-dihydro-17-hydroxy-3-oxo-, monopotassium salt, (6α,7α,11α,17α)-;
3′H-cyclopropa(6,7)pregna-4,6-diene-21-carboxylic acid, 9,11-epoxy-6,7-dihydro-17-hydroxy-3-oxo-, γ-lactone, (6α,7α,11α,17α)-;
pregn-4-ene-7,21-dicarboxylic acid, 9,11-epoxy-17-hydroxy-3-oxo-,γ-lactone, ethyl ester, (6α,7α,11α,17α)-; and
pregn-4-ene-7,21-dicarboxylic acid, 9,11-epoxy-17-hydroxy-3-oxo-,γ-lactone, 1-methylethyl ester, (7α,11α,17α)-.

20. The method of claim 1 wherein the anti-obesity agent is selected from the group consisting of gastrointestinal lipase inhibitors, mixed norepinephrine and serotonin reuptake inhibitors, and the pharmaceutically acceptable salts, esters, conjugate acids, and prodrugs thereof.

21. The method of claim 20 wherein the aldosterone receptor antagonist is eplerenone.

22. The method of claim 2 wherein the anti-obesity agent is selected from the group consisting of orlistat, sibutramine, and pharmaceutically acceptable salts, esters, conjugate acids, and prodrugs thereof.

23. The method of claim 21 wherein the anti-obesity agent is orlistat.

24. The method of claim 21 wherein the anti-obesity agent is sibutramine.

25. The method of claim 1 wherein the aldosterone receptor antagonist is spironolactone.

26. The method of claim 1 wherein the anti-obesity agent is orlistat.

27. The method of claim 1 wherein the anti-obesity agent is sibutramine.

28. The method of claim 1 wherein the anti-obesity agent is selected from the group consisting of a ciliary neurotrophic factor or one of its related variants or analogs; an appetite suppressant; a 5-HT-2c receptor agonist; a cannabinoid antagonist; a peptide or nonpeptide agonist of the cholecystokinin-A receptor; a peripherally acting agent that delays gastric emptying and increases satiety; an agonist of GLP-1 receptor; GLP-ls and related analogs; and the pharmaceutically acceptable salts, esters, conjugate acids, and prodrugs thereof.

29. The method of claim 28 wherein the aldosterone receptor antagonist is eplerenone.

30. The method of claim 1 wherein the anti-obesity agent is selected from the group consisting of Axokine, CP-644673/P-57, BVT-933, Rimonabant/SR-141716, GI-181771, HMR-1426, and Exendin-4.

31. The method of claim 30 wherein the aldosterone receptor antagonist is eplerenone.

32. The method of claim 1 wherein the anti-obesity agent is selected from the group consisting of human growth hormone fragments; PTP-1B inhibitors; DPP-IV inhibitors; and the pharmaceutically acceptable salts, esters, conjugate acids, and prodrugs thereof.

33. The method of claim 32 wherein the aldosterone receptor antagonist is eplerenone.

34. The method of claim 1 wherein the anti-obesity agent is selected from the group consisting of neuropeptide Y1 antagonists; neuropeptide Y5 antagonists; thyroid hormone receptor beta agonists; glucocorticoid antagonists; melanocortin-4 receptor (MC-4) agonists;
adiponectin/APM1/acrp30 and related analogs; and the pharmaceutically acceptable salts, esters, conjugate acids, and prodrugs thereof.

35. The method of claim 34 wherein the aldosterone receptor antagonist is eplerenone.

36. The method of claim 1 the anti-obesity agent is selected from the group consisting of 1 1-beta-hydroxysteroid dehydrogenase-1 inhibitors; fatty acid synthase inhibitors; acetyl CoA carboxylase inhibitors, and the pharmaceutically acceptable salts, esters, conjugate acids, and prodrugs thereof.

37. The method of claim 36 wherein the aldosterone receptor antagonist is eplerenone.

38. The method of claim 1 wherein the aldosterone receptor antagonist and the anti-obesity agent are administered in a sequential manner.

39. The method of claim 1 wherein the aldosterone receptor antagonist and the neutral endopeptidase inhibitor are administered in a substantially simultaneous manner.

40. The method of claim 1 wherein the aldosterone receptor antagonist is administered in a daily dose ranging from about 0.1 to about 2000 mg, and the anti-obesity agent is administered in a daily dose ranging from about 0.1 to about 1000 mg.

41. The method of claim 40 wherein the aldosterone receptor antagonist is eplerenone.

42. The method of claim 41 wherein the eplerenone is provided in a daily dose ranging from about 1 to about 250 mg.

43. The method of claim 1 wherein the first amount of the aldosterone receptor antagonist produces no substantial diuretic or anti-hypertensive effect in a subject.

44. The method of claim 1 further comprising administering a third amount of a compound selected from the group consisting of anti-diabetic agents, renin inhibitors, angiotensin I antagonists, angiotensin II antagonists, angiotensin converting enzyme inhibitors, alpha-adrenergic receptor blockers, beta-adrenergic receptor blockers, calcium channel blockers, endothelin receptor antagonists, endothelin converting enzymes, vasodilators, diuretics, cyclooxygenase-2 inhibitors, apical sodium bile acid transport inhibitors, cholesterol absorption inhibitors, fibrates, niacin, statins, cholesteryl ester transfer protein inhibitors, bile acid sequestrants, anti-oxidants, vitamin E, probucol, IIb/IIIa antagonists, xemilofiban, and orbofiban.

45. A pharmaceutical composition comprising a first amount of an aldosterone receptor antagonist, a second amount of an anti-obesity agent, and a pharmaceutically acceptable carrier, wherein the first amount of the aldosterone receptor antagonist and the second amount of the anti-obesity agent together comprise a therapeutically-effective amount of the aldosterone receptor antagonist and anti-obesity agent.

46. The composition of claim 45 wherein the aldosterone receptor antagonist is selected from the group consisting of:
pregn-4-ene-7,21-dicarboxylic acid, 9,11-epoxy-17-hydroxy-3-oxo,γ-lactone, methyl ester, (7α,11α,17α)-;
pregn-4-ene-7,21-dicarboxylic acid, 9,11-epoxy-17-hydroxy-3-oxo-dimethyl ester,(7α,11α,17α)-;
3′H-cyclopropa(6,7) pregna-4,6-diene-21-carboxylic acid, 9,11-epoxy-6,7-dihydro-17-hydroxy-3-oxo-,γ-lactone, (61,71,110,171)-;
pregn-4-ene-7,21-dicarboxylic acid, 9,11-epoxy-17-hydroxy-3-oxo-,7-(1-methylethyl) ester, monopotassium salt,(7α,11α,17α)-;
pregn-4-ene-7,21-dicarboxylic acid, 9,11,-epoxy-17-hydroxy-3-oxo-,7-methyl ester, monopotassium salt, (7α,11α,17α)-;
3′H-cyclopropa(6,7)pregna-1,4,6-triene-21-carboxylic acid, 9,11-epoxy-6,7-dihydro-17-hydroxy-3-oxo-, g-actone(6α,7α,11α)-;
3′H-cyclopropa(6,7)pregna-4,6-diene-21-carboxylic acid, 9,11-epoxy-6,7-dihydro-17-hydroxy-3-oxo-, methyl ester, (6α,7α,11α,17α)-;
3′H-cyclopropa(6,7)pregna-4,6-diene-21-carboxylic acid, 9,11-epoxy-6,7-dihydro-17-hydroxy-3-oxo-, monopotassium salt, (6α,7α,11α,17α)-;
3′H-cyclopropa(6,7)pregna-4,6-diene-21-carboxylic acid, 9,11-epoxy-6,7-dihydro-17-hydroxy-3-oxo-, g-lactone, (6α,7α,11α,17α)-;
pregn-4-ene-7,21-dicarboxylic acid, 9,11-epoxy-17-hydroxy-3-oxo-, g-lactone, ethyl ester, (7α,11α,17α)-; and
pregn-4-ene-7,21-dicarboxylic acid, 9,11-epoxy-17-hydroxy-3-oxo-, g-lactone, 1-methylethyl ester, (7α,1α,17α)-.

47. The composition of claim 45 wherein the aldosterone receptor antagonist is eplerenone.

48. The composition of claim 47 wherein the eplerenone is administered in a daily dose range from about 1 to about 250 mg.

49. The composition of claim 45 wherein the anti-obesity agent is selected from the group consisting of gastrointestinal lipase inhibitors, mixed norepinephrine and serotonin reuptake inhibitors, and the pharmaceutically acceptable salts, esters, conjugate acids, and prodrugs thereof.

50. The composition of claim 49 wherein the aldosterone receptor antagonist is eplerenone.

51. The composition of claim 45 wherein the anti-obesity agent is selected from the group consisting of orlistat and sibutramine and the pharmaceutically acceptable salts, esters, conjugate acids, and prodrugs thereof.

52. The composition of claim 51 wherein the aldosterone receptor antagonist is eplerenone.

53. The composition of claim 45 wherein the anti-obesity agent is selected from the group consisting of a ciliary neurotrophic factor or one of its related variants or analogs; an appetite suppressant; a 5-HT-2c receptor agonist; a cannabinoid antagonist; a peptide or nonpeptide agonist of the cholecystokinin-A receptor; a peripherally acting agent that delays gastric emptying and increases satiety; an agonist of GLP-1 receptor; GLP-1s and related analogs, and the pharmaceutically acceptable salts, esters, conjugate acids, and prodrugs thereof.

54. The composition of claim 53 wherein the aldosterone receptor antagonist is eplerenone.

55. The composition of claim 45 wherein the anti-obesity agent is selected from the group consisting of Axokine, CP-644673/P-57, BVT-933, Rimonabant/SR-141716, GI-181771, HMR-1426, and Exendin-4.

56. The composition of claim 55 wherein the aldosterone receptor antagonist is eplerenone.

57. The composition of claim 45 wherein the anti-obesity agent is selected from the group consisting of human growth hormone fragments; PTP-1B inhibitors; DPP-IV inhibitors; and the pharmaceutically acceptable salts, esters, conjugate acids, and prodrugs thereof.

58. The composition of claim 57 wherein the aldosterone receptor antagonist is eplerenone.

59. The composition of claim 45 wherein the anti-obesity agent is selected from the group consisting of neuropeptide Y1 antagonists; neuropeptide Y5 antagonists; thyroid hormone receptor beta agonists; glucocorticoid antagonists; melanocortin-4 receptor (MC-4) agonists; adiponectin/APM1/acrp30 and related analogs, and the pharmaceutically acceptable salts, esters, conjugate acids, and prodrugs thereof.

60. The composition of claim 59 wherein the aldosterone receptor antagonist is eplerenone.

61. The composition of claim 45 the anti-obesity agent is selected from the group consisting of 11-beta-hydroxysteroid dehydrogenase-1 inhibitors; fatty acid synthase inhibitors; acetyl CoA carboxylase inhibitors; and the pharmaceutically acceptable salts, esters, conjugate acids, and prodrugs thereof.

62. The composition of claim 61 wherein the aldosterone receptor antagonist is eplerenone.

63. The composition of claim 45 wherein the first amount of the aldosterone receptor antagonist produces no substantial diuretic or anti-hypertensive effect in a subject.

64. The composition of claim 45 further comprising a third amount of a compound selected from the group consisting of anti-diabetic agents, renin inhibitors, angiotensin I antagonists, angiotensin II antagonists, angiotensin converting enzyme inhibitors, alpha-adrenergic receptor blockers, beta-adrenergic receptor blockers, calcium channel blockers, endothelin receptor antagonists, endothelin converting enzymes, vasodilators, diuretics, cyclooxygenase-2 inhibitors, apical sodium bile acid transport inhibitors, cholesterol absorption inhibitors, fibrates, niacin, statins, cholesteryl ester transfer protein inhibitors, bile acid sequestrants, anti-oxidants, vitamin E, probucol, IIb/IIIa antagonists, xemilofiban, and orbofiban.

65. A kit containing a first amount of an aldosterone receptor antagonist and a second amount of an anti-obesity agent.

66. The kit of claim 65 comprising the first amount of the aldosterone receptor antagonist in a unit dosage form, and the second amount of an anti-obesity agent in a unit dosage form.

67. The kit of claim 65 wherein the aldosterone receptor antagonist is eplerenone.

68. The kit of claim 65 wherein the anti-obesity agent is selected from the group consisting of gastrointestinal lipase inhibitors, mixed norepinephrine and serotonin reuptake inhibitors, and the pharmaceutically acceptable salts, esters, conjugate acids, and prodrugs thereof.

69. The kit of claim 68 wherein the aldosterone receptor antagonist is eplerenone.

70. The kit of claim 65 wherein the anti-obesity agent is selected from the group consisting of orlistat and sibutramine and the pharmaceutically acceptable salts, esters, conjugate acids, and prodrugs thereof.

71. The kit of claim 70 wherein the aldosterone receptor antagonist is eplerenone.

72. The kit of claim 65 wherein the anti-obesity agent is selected from the group consisting of a ciliary neurotrophic factor or one of its related variants or analogs; an appetite suppressant; a 5-HT-2c receptor agonist; a cannabinoid antagonist; a peptide or nonpeptide agonist of the cholecystokinin-A receptor; a peripherally acting agent that delays gastric emptying and increases satiety; an agonist of GLP-1 receptor; GLP-ls and related analogs;
and the pharmaceutically acceptable salts, esters, conjugate acids, and prodrugs thereof.

73. The kit of claim 72 wherein the aldosterone receptor antagonist is eplerenone.

74. The kit of claim 65 wherein the anti-obesity agent is selected from the group consisting of Axokine, CP-644673/P-57, BVT-933, Rimonabant/SR-141716, GI-181771, HMR-1426, and Exendin-4.

75. The kit of claim 74 wherein the aldosterone receptor antagonist is eplerenone.

76. The kit of claim 65 wherein the anti-obesity agent is selected from the group consisting of human growth hormone fragments; PTP-1B inhibitors; DPP-IV inhibitors;
and the pharmaceutically acceptable salts, esters, conjugate acids, and prodrugs thereof.

77. The kit of claim 76 wherein the aldosterone receptor antagonist is eplerenone.

78. The kit of claim 65 wherein the anti-obesity agent is selected from the group consisting of neuropeptide Y1 antagonists; neuropeptide Y5 antagonists; thyroid hormone receptor beta agonists; glucocorticoid antagonists; melanocortin-4 receptor (MC-4) agonists; adiponectin/APM1/acrp30 and related analogs; and the pharmaceutically acceptable salts, esters, conjugate acids, and prodrugs thereof.

79. The kit of claim 78 wherein the aldosterone receptor antagonist is eplerenone.

80. The kit of claim 65 the anti-obesity agent is selected from the group consisting of 11-beta-hydroxysteroid dehydrogenase-1 inhibitors; fatty acid synthase inhibitors; acetyl CoA carboxylase inhibitors; and the pharmaceutically acceptable salts, esters, conjugate acids, and prodrugs thereof.

81. The kit of claim 80 wherein the aldosterone receptor antagonist is eplerenone.

82. The kit of claim 65 wherein the aldosterone receptor antagonist is spironolactone.

83. The kit of claim 65 wherein the anti-obesity agent is selected from the group consisting of orlistat and sibutramine and the pharmaceutically acceptable salts, esters, conjugate acids, and prodrugs thereof.

84. The method of claim 44 wherein the angiotensin converting enzyme inhibitor is selected from the group consisting of benazapril; captopril; cilazapril; enalapril; fosinopril; lisinopril; perindopril; quinopril; ramipril; trandolapril; and the pharmaceutically acceptable salts, esters, conjugate acids, and prodrugs thereof.

85. The method of claim 84 wherein the anti-obesity agent is selected from the group consisting of orlistat and sibutramine, and the pharmaceutically acceptable salts, esters, conjugate acids, and prodrugs thereof.

86. The method of claim 85 wherein the aldosterone receptor antagonist is eplerenone.

87. The method of claim 44 wherein the anti-diabetic agent is metformin.
